(19) United States
(12) Patent Application Publication
(10) Pub. No.: US 2011/0117602 A1
(43) Pub. Date: May 19, 2011

(54) HUMAN ANTIBODY CAPABLE OF INDUCING APOPTOSIS

(75) Inventors: Yuji Ito, Kagoshima-ken (JP); Masanori Baba, Kagoshima-ken (JP); Naomichi Arima, Kagoshima-ken (JP); Yasuo Suda, Kagoshima-ken (JP); Toshihiro Nakashima, Kumamoto-ken (JP); Masaharu Torikai, Kumamoto-ken (JP)

(73) Assignees: KAGOSHIMA UNIVERSITY,, KAGOSHIMA-KEN (JP); JURIDICAL FDN THE CHEMO-SERO-THERAPEUTIC RES INST, KUMAMOTO-KEN (JP)

(21) Appl. No.: 12/743,739

(22) PCT Filed: Nov. 18, 2008
(86) PCT No.: PCT/JP08/70929

§ 371 Date: Jul. 12, 2010

(30) Foreign Application Priority Data
Nov. 19, 2007 (JP) 2007-299551

(51) Int. Cl. C07K 16/30; C07H 21/04; C12N 15/63; C12P 21/00

(52) U.S. Cl. 435/69.6; 530/387.3; 536/23.53; 435/320.1

(57) ABSTRACT

A human antibody that has a specific reactivity with multiple tumor cell lines including ATL cells and possesses both safety and therapeutic efficacy and a fragment of said antibody are provided. A human antibody and a fragment of said antibody that may recognize HLA-DR β chain expressed on the surface of tumor cells were obtained. In particular, it was found that a dimer of scFv (diabody) of said antibody may induce potent apoptosis in cells expressing the HLA-DR β chain. The antibody and a fragment of said antibody obtained in accordance with the present invention are useful for a detection reagent, a diagnostic and a medicament for protection or treatment of cancers including ATL and/or viral infectious diseases.

### TECHNICAL FIELD

The present invention relates to a human antibody that targets Human Leukocyte Antigen (HLA)-DR β chain, a cell surface molecule highly expressed on tumor cells, and induces cell death (apoptosis) in cells highly expressing HLA-DR β chain, and a fragment of said antibody.

### BACKGROUND ART

An antibody medicine is establishing a position of an efficacious medicament to a variety of diseases. An antibody medicine, as being a biomedicine comprising a molecule derived from a living organism, has less adverse side effects such as toxicity. Since an antibody is one of the most important molecules involved in specific recognition of a foreign substance in the immune system, an antibody medicine, as prepared from such an antibody, is also one of molecular targeting medicines taking in part a high level of molecular recognition. In particular, for cancers, anti-CD20 antibody (rituximab) against B cell lymphoma and anti-HER2 antibody (herceptin) against breast cancer have been used for therapy to prove their efficaciousness. A principal mechanism of the therapeutic effect is supposed to be that, as a consequence of administration of an antibody to a cancer-specific antigen (cancer marker), specific immunological induction occurs by NK (Natural Killer) cells attacking cancer cells to which antibodies are bound and aggregated, i.e. Antibody-Dependent Cell-mediated Cytotoxicity (ADCC) or Complement-Dependent Cytotoxicity (CDC), to thereby lead the cancer cells to apoptosis.

At present, numerous antibody medicines have been developed for various diseases including cancers. Process for preparing an antibody includes three approaches. The first is to prepare a mouse antibody using the conventional mouse hybridoma method to prepare a mouse antibody which is then humanized by antibody engineering. However, a humanized antibody partially contains a non-human sequence and thus its repetitive or long-term administration may induce production of an antibody that inhibits the activity of the humanized antibody as administered to thereby not only reduce efficacy extremely but also induce severe side effects. Besides, the activity may sometimes be reduced as a consequence of humanization and thus construction of a humanized antibody may require a vast labor and cost.

The other processes for preparing an antibody include approach with hybridoma technique using a so-called trans-chromosomic mouse where a portion of human chromosomes is incorporated and an antibody phage library method. It is anticipated that the use of these two approaches will be accelerated in the future since they allow for direct isolation of a human antibody.

ATL (adult T cell leukemia) is a disease caused by a retrovirus, a human T-lymphotropic virus type 1 (HTLV-1) (Non-patent reference 1). HTLV-1, mostly transmitted by mother-to-child transmission through mother's milk, is reported to have 1.2 million carriers in Japan but its distribution is biased to Southern Kyushu area including Kagoshima. ATL usually develops after a long latent period but not in all of carriers with its onset rate being around 6.6% for men and around 2.1% for women. It is also sometimes accompanied by HAM (HLLV-1 associated myelopathy), a neurodegenerative disease caused by HTLV-1, upon whose development, prognosis is bad and no efficacious therapy bas hitherto been established (Non-patent reference 2). There is no report on a monoclonal antibody that specifically binds to cells infected with HTLV-1.

On the other hand, for cells of other lymphoma or leukemia, Major Histocompatibility Complex Class II (MHC Class II) is known as one of highly expressed molecules. MHC Class II, a molecule acting as a signal between lymphocytes and antigen presenting cells, is expressed only in immunological cells (B lymphocytes, activated T lymphocytes, macrophages and dendritic cells) under physiological conditions but, as reported, also in various cells through cytokine induction in case of certain diseases (tumoral diseases, autoimmune diseases and infectious diseases).

For an anti-MHC Class II antibody, it is reported that the antibody induces caspase-independent apoptosis in cells of other lymphoma or leukemia (Non-patent references 3 to 6). The most likely action mechanism is one through activation of intracellular Protein kinase C by Apoptosis inducing factor (Non-patent reference 7). For HLA-DR, a human MHC Class II, is reported that a humanized antibody from a mouse anti-HLA-DR antibody 1D10 (Non-patent reference 8), a human anti-HLA-DR antibody obtained from a human synthetic antibody phage library (HuCAL) (Non-patent reference 9) and, recently, a human anti-HLA-DR antibody isolated by a hybridoma technique from a trans-chromosomic mouse immunized with HLA-DR-transfected L929 cells (Non-patent reference 10) induced apoptosis in cells of various lymphoma or leukemia. However, most of these are experimented with antibodies in the form of an intact molecule.

For an antibody in the form of an intact molecule, there are accumulated findings on their property and function as a consequence of research for many years and it conferred major achievements in an antibody medicine. A long half-life of an antibody in the form of an intact molecule as long as several weeks or more is thought to be one of its advantages. However, for use as an agonist antibody such that an anti-MHC Class II antibody is used to induce apoptosis, a long half-life would become possibly disadvantageous. Indeed, by way of example, in Phase I clinical test for an anti-CD28 antibody to be used as an agonist antibody for treating autoimmune diseases, severe adverse side effects were observed to discontinue the clinical test (TGN1412). As such, for an agonist antibody, a molecular form with which a half-life is controllable would possibly be more useful.

An antibody with a lower molecular weight is characterized by its easy molecular alteration or modification through protein engineering technique. It is known that, in general, although an antibody with a lower molecular weight per se has a quite short half-life in blood, a half-life in blood may be prolonged by modification with polyethylene glycol (PEG), or by fusion with albumin or fusion with a sequence that binds to albumin or IgG.

An antibody in the form of an intact molecule may exert ADCC activity or CDC activity via Fc domain. These activities may be efficacious for target cells but may possibly be detrimental to cells other than target cells to cause adverse side effects as apprehended. In this regard, in case of an antibody with a lower molecular weight, not having Fc domain, possibility of side effects due to ADCC activity or CDC activity may be denied since only the activity of variable regions is taken into consideration.

Besides, it is also contemplated that an antibody with a lower molecular weight as a medicament may be used more advantageously than an antibody in the form of an intact molecule for some usages, e.g. possessing a higher productivity or more excellent invasiveness into tissues due to its small size.

As described above, if a human antibody with a lower molecular weight could be created, an antigen of which is HLA-DR and which has a potent apoptotic activity, the antibody would become an antibody medicine useful for cancer therapy. However, there is no report for such an antibody.
Non-patent reference 1: Tsukasaki, K. et al., Bailliere's Best Practice & Research 13, p. 231-243 (2000).
Non-patent reference 2: Ohshima, K. et al., Cancer Science 98, p. 772-778 (2007).
Non-patent reference 3: Newell, M. K. et al., Proceedings of the National Academy of Sciences of the United States of America 90, p. 10459-10463 (1993).
Non-patent reference 4: Bertho, N. et al., J Immunol 164, p. 2379-2385 (2000).
Non-patent reference 5: Al-Daccak, R. et al., Current Opinion in Immunology 16, p. 108-113 (2004).
Non-patent reference 6: Nagy, Z. A. & Mooney, N. A., Journal of Molecular Medicine-Jmm 81, p. 757-765 (2003).
Non-patent reference 7: Bertho, N. et al., International Immunology 14, p. 935-942 (2002).
Non-patent reference 8: Kostelny, S. A. et al., International Journal of Cancer 93, p. 556-565 (2001).
Non-patent reference 9: Nagy, Z. A. et al., Nature Medicine 8, p. 801-807 (2002).
Non-patent reference 10: Tawara, T. et al., Cancer Science 98, p. 921-928 (2007).

### DISCLOSURE OF THE INVENTION

### Technical Problem to be Solved by the Invention

The present invention provides a human antibody that has a specific reactivity with multiple tumor cell lines including ATL cells and possesses both safety and therapeutic efficacy, and a fragment of said antibody, and proposes a method for using the same.

### Means for Solving the Problems

Based on the findings as described above, aiming at creation of an antibody medicine for establishing a novel therapy for cancer such as ATL, the present inventors have succeeded in isolating a human antibody specific to S1T, cell strain from ATL patients, by screening from single-chain Fv (scFv) human antibody phage library as previously constructed. As a result of analysis of the obtained antibody, its antigen was found to be HLA-DR β chain known to be highly expressed also in cells of other lymphoma or leukemia. Interestingly, a dimer (diabody) of the obtained scFv human antibody was found to have an extremely high ability to induce apoptosis in S1T. Accordingly, it is presumed that the antibody of the present invention would also have a selective reactivity and an apoptosis-inducing ability to other cells highly expressing HLA-DR β chain.

The human antibody diabody obtained by the present invention was found to show an apoptosis-inducing ability at a much lower concentration than that of the anti-MHC Class II antibodies hitherto reported. Moreover, the activity was proved firstly for an anti-MHC Class II antibody in the format of a human antibody with a lower molecular weight and thus a possibility could be shown of the antibody of the present invention for use as an anti-cancer agent targeting MHC Class II molecule with excellent safety, productivity and invasiveness into tissues.

Thus, the present invention encompasses the inventions (1) to (29) as described below for methods and materials that may be useful from medical and industrial point of view.

(1) A human anti-HLA-DR β chain antibody comprising complementarity determining region (CDR) of an H chain that consists of a polypeptide (a) or (b) below and complementarity determining region (CDR) of an L chain that consists of a polypeptide (c) or (d) below, or a fragment of said antibody:

(a) a polypeptide that has the amino acid sequences as depicted in SEQ ID NO: 2 for CDR1, SEQ ID NO: 3 for CDR2, and SEQ ID NO: 4 for CDR3;

(b) a polypeptide that has the amino acid sequences as depicted in SEQ ID NOs: 2-4 with substitution, deletion, insertion and/or addition of one or more amino acid residues that may serve as complementarity determining region of an H chain of an antibody that recognizes HLA-DR β chain and has an apoptosis-inducing ability in cells expressing said HLA-DR β chain;

(c) a polypeptide that has the amino acid sequences as depicted in SEQ ID NO: 7 for CDR1, SEQ ID NO: 8 for CDR2, and SEQ ID NO: 9 for CDR3;

(d) a polypeptide that has the amino acid sequences as depicted in SEQ ID NOs: 7-9 with substitution, deletion, insertion and/or addition of one or more amino acid residues that may serve as complementarity determining region of an L chain of an antibody that recognizes HLA-DR β chain and has an apoptosis-inducing ability in cells expressing said HLA-DR β chain.

(2) The human anti-HLA-DR β chain antibody or a fragment of said antibody according to (1) above wherein the polypeptides (b) and (d) have amino acid homology of about 90%, more preferably about 95%, most preferably about 97%, about 98% or about 99% or more to the amino acid sequences before subject to substitution, deletion, insertion and/or addition of amino acid residues.

(3) The human anti-HLA-DR β chain antibody or a fragment of said antibody according to (1) above wherein said antibody comprises an H chain variable region consisting of a polypeptide (e) or (f) below and an L chain variable region consisting of a polypeptide (g) or (h) below:

(e) a polypeptide consisting of the amino acid sequence as depicted in SEQ ID NO: 1;

(f) a polypeptide consisting of the amino acid sequences as depicted in SEQ ID NO: 1 with substitution, deletion, insertion and/or addition of one or more amino acid residues that may serve as an H chain variable region of an antibody that recognizes HLA-DR β chain and has an apoptosis-inducing ability in cells expressing said HLA-DR β chain;

(g) a polypeptide consisting of the amino acid sequence as depicted in SEQ ID NO: 6;

(h) a polypeptide consisting of the amino acid sequences as depicted in SEQ ID NO: 6 with substitution, deletion, insertion and/or addition of one or more amino acid residues that may serve as an L chain variable region of an antibody that recognizes HLA-DR β chain and has an apoptosis-inducing ability in cells expressing said HLA-DR β chain.

(4) The human anti-HLA-DR β chain antibody or a fragment of said antibody according to (3) above wherein the polypeptides (f) and (h) have amino acid homology of about 90%, more preferably about 95%, most preferably about 97%, about 98% or about 99% or more to the amino acid sequences before subject to substitution, deletion, insertion and/or addition of amino acid residues.

(5) The human anti-HLA-DR β chain antibody or a fragment of said antibody according to any of (1) to (4) above wherein said fragment is Fab, Fab′, F(ab′)₂, scAb, scFv, (scFv)₂, scFv dimer (diabody) or other antibody fragments with two or more scFv being bound, or Fc fusion thereof.

(6) The human anti-HLA-DR β chain antibody or a fragment of said antibody according to (5) above wherein said fragment is scFv or scFv-Fc.

(7) The human anti-HLA-DR β chain antibody or a fragment of said antibody according to (5) above wherein said fragment is diabody, or Fc fusion with said diabody.

(8) An H chain variable region fragment of a human anti-HLA-DR β chain antibody consisting of a polypeptide comprising complementarity determining region (CDR) of an H chain consisting of a polypeptide (a) or (b) below:

(a) a polypeptide that has the amino acid sequences as depicted in SEQ ID NO: 2 for CDR1, SEQ ID NO: 3 for CDR2, and SEQ ID NO: 4 for CDR3;

(b) a polypeptide that has the amino acid sequences as depicted in SEQ ID NOs: 2-4 with substitution, deletion, insertion and/or addition of one or more amino acid residues that may serve as complementarity determining region of an H chain of an antibody that recognizes HLA-DR β chain and has an apoptosis-inducing ability in cells expressing said HLA-DR β chain.

(9) The H chain variable region fragment according to (8) above wherein the polypeptide (b) has amino acid homology of about 90%, more preferably about 95%, most preferably about 97%, about 98% or about 99% or more to the amino acid sequences before subject to substitution, deletion, insertion and/or addition of amino acid residues.

(10) The H chain variable region fragment according to (8) above wherein the polypeptide comprising CDR of said H chain is a polypeptide (e) or (f) below:

(e) a polypeptide consisting of the amino acid sequence as depicted in SEQ ID NO: 1;

(f) a polypeptide consisting of the amino acid sequences as depicted in SEQ ID NO: 1 with substitution, deletion, insertion and/or addition of one or more amino acid residues that may serve as an H chain variable region of an antibody that recognizes HLA-DR β chain and has an apoptosis-inducing ability in cells expressing said HLA-DR β chain.

(11) The H chain variable region fragment according to (10) above wherein the polypeptide (f) has amino acid homology of about 90%, more preferably about 95%, most preferably about 97%, about 98% or about 99% or more to the amino acid sequences before subject to substitution, deletion, insertion and/or addition of amino acid residues.

(12) An L chain variable region fragment of a human anti-HLA-DR β chain antibody consisting of a polypeptide comprising complementarity determining region (CDR) of an L chain consisting of a polypeptide (c) or (d) below:

(c) a polypeptide that has the amino acid sequences as depicted in SEQ ID NO: 7 for CDR1, SEQ ID NO: 8 for CDR2, and SEQ ID NO: 9 for CDR3;

(d) a polypeptide that has the amino acid sequences as depicted in SEQ ID NOs: 7-9 with substitution, deletion, insertion and/or addition of one or more amino acid residues that may serve as complementarity determining region of an L chain of an antibody that recognizes HLA-DR β chain and has an apoptosis-inducing ability in cells expressing said HLA-DR β chain.

(13) The L chain variable region fragment according to (12) above wherein the polypeptide (d) has amino acid homology of about 90%, more preferably about 95%, most preferably about 97%, about 98% or about 99% or more to the amino acid sequences before subject to substitution, deletion, insertion and/or addition of amino acid residues.

(14) The L chain variable region fragment according to (12) above wherein the polypeptide comprising CDR of said L chain is a polypeptide (g) or (h) below:

(g) a polypeptide consisting of the amino acid sequence as depicted in SEQ ID NO: 6;

(h) a polypeptide consisting of the amino acid sequences as depicted in SEQ ID NO: 6 with substitution, deletion, insertion and/or addition of one or more amino acid residues that may serve as an L chain variable region of an antibody that recognizes HLA-DR β chain and has an apoptosis-inducing ability in cells expressing said HLA-DR β chain.

(15) The L chain variable region fragment according to (14) above wherein the polypeptide (h) has amino acid homology of about 90%, more preferably about 95%, most preferably about 97%, about 98% or about 99% or more to the amino acid sequences before subject to substitution, deletion, insertion and/or addition of amino acid residues.

(16) A human anti-HLA-DR β chain antibody fragment which is formed by binding together the H chain variable region fragment according to any of (8) to (11) above and the L chain variable region fragment according to any of (12) to (15) above and which is in the form of scFv, (scFv)₂, diabody, or other antibody fragments with two or more scFv being bound.

(17) The human anti-HLA-DR β chain antibody fragment according to (16) above wherein said antibody fragment is scFv.

(18) The human anti-HLA-DR β chain antibody fragment according to (16) above wherein said antibody fragment is diabody.

(19) A human anti-HLA-DR β chain antibody or a fragment of said antibody which is formed by binding a constant region of a human-derived antibody to the H chain variable region fragment according to any of (8) to (11) above and/or to the L chain variable region fragment according to any of (12) to (15) above.

(20) The human anti-HLA-DR β chain antibody or a fragment of said antibody according to (19) above wherein said fragment is Fc fusion with Fab, Fab′, F(ab′)₂, scAb, scFv, (scFv)₂, scFv dimer (diabody) or Fc fusion with other antibody fragments with two or more scFv being bound.

(21) A fused antibody comprising the antibody or a fragment of said antibody according to any of (1) to (20) above fused to a peptide or other protein.

(22) A modified antibody which is formed by binding the antibody or a fragment of said antibody according to any of (1) to (20) above or the fused antibody according to (21) above to a modifying agent.

(23) A gene coding for the antibody or a fragment of said antibody according to any of (1) to (20) above or the fused antibody according to (21) above.

(24) A recombinant expression vector comprising the gene according to (23) above.

(25) A transfectant with the gene according to (23) above introduced therein.

(26) A method for producing a human anti-HLA-DR β chain antibody or a fragment of said antibody by expressing the gene according to (23) above in a host cell.

(27) A reagent for detection and/or a diagnosis of cancers and/or viral infectious diseases such as ATL comprising the antibody or a fragment of said antibody according to any of (1) to (20) above, the fused antibody according to (21) above or the modified antibody according to (22) above.

(28) A protective or therapeutic agent for cancers and/or viral infectious diseases such as ATL comprising the antibody or a fragment of said antibody according to any of (1) to (20) above, the fused antibody according to (21) above or the modified antibody according to (22) above.

(29) A use of the antibody or a fragment of said antibody according to any of (1) to (20) above, the fused antibody according to (21) above or the modified antibody according to (22) above in the manufacture of a medicament for preventing or treating cancers and/or viral infectious diseases such as ATL.

### Effects of the Invention

The human monoclonal antibody and a fragment of said antibody of the present invention may target HLA-DR β chain, the cell surface molecule highly expressed on tumor cells such as ATL and induce apoptosis in cells expressing HLA-DR β chain.

An antibody medicine is extremely liable to practical usage due to its apparent targeting mechanism and action mechanism facilitating thereby process for practical application of medicinal development. HLA-DR β Chain recognized by the antibody of the present invention is likely to be a good marker of ATL or other cancers and thus there is a possibility that establishment of therapy could be attained relatively easily to prove that the present invention is of highly industrial utility. In addition, the human monoclonal antibody and a fragment of said antibody of the present invention may also possibly be a medicament efficacious for immunological diseases caused by abnormality of the immune system or various infectious diseases.

### BRIEF DESCRIPTION OF DRAWINGS

FIG. 1 is a scheme showing a flow of bio-panning technique for obtaining an antibody phage targeting specific cells with a flow cytometer using cellular fluorescent labeling.

FIG. 2 shows the results of analysis of a mixture of antibody phages obtained from each of 1-3 rounds of panning for their reactivity with S1T cells with a flow cytometer.

FIG. 3 shows the results of analysis of antibody phage clones obtained from each of 1-3 rounds of panning for their reactivity with S1T cells with a flow cytometer.

FIG. 4 shows an amino acid sequence of scFv antibody phage clone (MS-S1TA3) specifically binding to S1T cells.

FIG. 5 shows the results of FACS analysis for binding property of purified scFv antibody (MS-S1TA3) to S1T cells. A: Cells alone; B: Stained with mouse anti-His antibody+mouse IgG antibody bound with FITC; C: Stained with MS-S1TA3 scFv+mouse anti-His antibody+mouse IgG antibody bound with FITC.

FIG. 6 shows the results of analysis for comparing binding property of scFv antibody (MS-S1TA3) to cell lines infected with and without HTLV-1. A: Cells alone; B: Stained with mouse anti-His antibody+mouse IgG antibody bound with FITC; C: Stained with MS-S1TA3 scFv+mouse anti-His antibody+mouse IgG antibody bound with FITC.

FIG. 7 shows the results of Western blotting analysis for the reactivity of scFv antibody (MS-S1TA3) to lysate of S1T cells.

FIG. 8 shows binding activity of scFv antibody (MS-S1TA3) to L cell transformant strain (L57.23) expressing HLA-DR β chain (DRB1*0101) and HLA-DR α chain (DRA*0101). For L243, A: Cells alone; B: Stained with anti-HLA-DR antibody labeled with PE. For MS-S1TA3, A: Cells alone; B: Stained with mouse anti-His antibody+mouse IgG antibody bound with FITC; C: Stained with MS-S1TA3 scFv+mouse anti-His antibody+mouse IgG antibody bound with FITC.

FIG. 9 shows binding property of scFv antibody (MS-S1TA3) to a recombinant HLA-DR molecule.

FIG. 10 shows that addition of scFv antibody (MS-S1TA3) induces apoptosis in S1T cells.

FIG. 11 shows induction of apoptosis in S1T cells by L243 mouse monoclonal antibody.

FIG. 12 shows induction of apoptosis in S1T cells by a monomer of scFv antibody (MS-S1TA3) with linkers of different length.

FIG. 13 shows induction of apoptosis in S1T cells by a dimer (diabody) of scFv antibody (MS-S1TA3) with linkers of different length.

FIG. 14 shows structure of various derivatives of MS-S1TA3.

FIG. 15 shows the results of ELISA analysis for the binding activity of scFv-Fc antibody (MS-S1TA3) to HLA-DR.

FIG. 16 shows the results of FACS analysis for binding property of scFv-Fc antibody (MS-S1TA3) to S1T cells. A: Cells alone; B: Stained with control IgG antibody+anti-human Fc antibody labeled with biotin+streptavidin labeled with PE; C: Stained with MS-S1TA3 scFv-Fc+anti-human Fc antibody labeled with biotin+streptavidin labeled with PE.

FIG. 17 shows induction of apoptosis in S1T cells by scFv-Fc antibody (MS-S1TA3) in comparison with other forms.

FIG. 18 shows induction of apoptosis in cells of various lymphoma or leukemia by scFv antibody (MS-S1TA3) diabody.

FIG. 19 shows difference in HLA-DR expression in cells of various lymphoma or leukemia wherein the figures in parenthesis indicate a median of florescent intensity in a population of cells. A: Cells alone; B: Stained with anti-HLA-DR antibody labeled with PE.

### BEST MODE FOR CARRYING OUT THE INVENTION

The present invention is explained in detail hereinbelow. scFv Display phage library may be prepared as described below. From peripheral blood B lymphocytes taken from plural healthy donors, immunoglobulin heavy (H) chain and light (L) chain cDNAs are synthesized by RT-PCR. Next, by using a combination of various primers, an H chain variable region (VH) and an L chain variable region (VL) are amplified and linked together with a linker DNA to produce scFv gene library of random combination of VH and VL from lymphocytes of healthy donors. The scFv genes are incorporated into phagemid vector pCANTAB5E to construct scFv display phage library comprising about 10⁸ to 10¹¹ clones from healthy donors.

Approach for isolating a specific antibody using antibody phage library is called “panning” and includes various modes as reported. Most generally, in case that a targeting antigen is known, the antigen is prepared as a recombinant protein, which is immobilized on a plastic plate to which an antibody phage library is added for reaction to thereby concentrate a specifically binding antibody phage. However, for isolating an antibody phage specific to a cell line when a targeting antigenic protein is not known as in the present invention, panning with cells per se and flow cytometer is possible. Alternatively, instead of cells per se, a membrane fraction may be prepared and used as an antigen.

For instance, an antibody phage library is added to a mixture of MOLT-4 cells as a control and S1T cells as a target cell previously labeled with FITC-labeled antibody or directly with FITC labeling agent, and the mixture is reacted at 4° C. for 1 hour while slowly stirring. After centrifugation, supernatant is removed and precipitated cells are washed. The precipitated cells after centrifugation are suspended in FACS buffer and S1T cells are isolated with a flow cytometer. Phages are eluted from the collected cells by adding an acidic solution and, after neutralization, infected to E. coli (TG1). The antibody phages are rescued with a helper phage and again subject to the same panning process. Panning processes are repeated for several times to concentrate specific clones.

The antibody phages obtained in each of the panning processes may be tested for their binding activity to S1T cells with a flow cytometer. As a consequence of estimation of their reactivity to S1T cells and MOLT-4 cells, if a shift of fluorescent intensity apparently attributable to the binding is observed only for S1T cells with repetition of the panning processes, then it is proved that antibody phages specifically binding to S1T cells are concentrated.

When the antibody phages are proved to be concentrated, the antibody phages at that panning process are infected to E. coli and, after cloning, each of the phage clones may be tested for their binding activity to S1T cells with a flow cytometer. As a result, if a specific reactivity to S1T cells was observed for these clones, these clones may be subject to DNA sequencing to determine DNA sequences encoding amino acid sequences of VH and VL regions of an antibody.

scFv of the clones thus obtained may be prepared and tested for their reactivity to target cells. For expression of scFv, it may be expressed e.g. in E. coli where a useful promoter as commonly used, a signal sequence for secretion of an antibody, and the like may functionally be bound. Promoter that may be used includes lacZ promoter, araB promoter, and the like. A signal sequence for secretion of scFv that may be used includes pelB signal sequence (J. Bacterio R 1987, 169: 4379-4383) for expression in periplasm of E. coli. For secretion in culture supernatant, a signal sequence of M13 phage g3 protein may also be used.

scFv expressed extracellularly or intracellularly may be isolated from host cells and purified to uniformity. Purification may easily be performed in short time by affinity chromatography with anti-Etag antibody when Etag sequence is attached at the C terminus of scFv or by affinity chromatography with NTA column when His-tag sequence is attached to scFv. It is also possible to employ a combination of isolation/purification procedures as used for ordinary proteins. For instance, ultrafiltration, salting-out, column chromatography such as gel filtration/ion exchange/hydrophobic chromatography may be used in combination to isolate and purify an antibody. A purified product may be analyzed for its molecular form with HPLC gel filtration, and the like.

The obtained purified product of scFv may be used to ascertain its binding activity to S1T cells by analysis with a flow cytometer.

Specificity of the obtained scFv antibody may preferably be analyzed with various HTLV-1-infected cell lines (e.g. S1T, MT-4, M8166 and MT-2 which are HTLV-1-infected T lymphomas) and with non-infected cell lines (MOLT-4, CEM, Jurkat and Daudi which are non-HTLV-1-infected lymphomas). If the scFv antibody is more likely to bind to HTLV-1-infected cell lines than non-HTLV-1-infected cell lines, it is suggested that said scFv antibody may be an antibody efficaciously targeting HTLV-1-infected T cell lymphomas.

For identifying an antigen for scFv antibody, a lysate of target cells may be subject to SDS-PAGE (under reduced condition) by Western blotting followed by immunostaining with scFv antibody. As a result, when bands are detected, they are cut from the gel after electrophoresis, reductive alkylation and trypsin digestion are carried out in the gel and the obtained peptide fragments are analyzed by LC-MS/MS analysis. From the obtained mass spectrum data of fragments, candidate proteins contained in the gel are narrowed down by MASCOT retrieval and, among proteins with high score, those on the cell surface may be selected as a candidate of an antigen.

For ascertaining the results, for instance, binding property to cells transfected with a gene encoding a protein of candidate antigen may be surveyed with a flow cytometer or alternatively reactivity with a purified protein of candidate antigen may be estimated by Western blotting or ELISA or Biacore. Based on these results, identification of an antigen for scFv antibody may be concluded.

A biological activity of the obtained scFv antibody may also be estimated. For instance, an apoptosis-inducing activity may be analyzed by mixing target cells with scFv antibody for reaction and then staining with FITC-labeled Annexin V and PI followed by analysis with a flow cytometer. If increase in doubly positive cells is observed with addition of scFv antibody in a manner specific to target cells, it is then suggested that said scFv antibody may have an apoptosis-inducing ability.

In general, it is reported that the format of scFv is such that a monomer and a dimer, i.e. a diabody, are present in admixture thereof (Kortt, A. A, 2001). Thus, for investigating which format is a molecular species responsible for induction of apoptosis, a monomer and a dimer may be separated from each other by molecular sieve chromatography and then compared for their apoptosis-inducing ability. Besides, from scFv where the linker is 15-mer in length (linker 3, (GGGGS)×3: SEQ ID NO: 15; the corresponding nucleotide sequence is shown in SEQ ID NO: 16) may be prepared scFv where the linker is 10-mer in length (linker 2, (GGGGS)×2: SEQ ID NO: 13; the corresponding nucleotide sequence is shown in SEQ ID NO: 14) and scFv where the linker is 5-mer in length (linker 1, (GGGGS)×1; SEQ ID NO: 11; the corresponding nucleotide sequence is shown in SEQ ID NO: 12) and a monomer and a dimer of the respective scFv may be compared with each other for their apoptosis-inducing ability.

A human antibody specific to S1T cells, a cell line derived from ATL cells, was screened from human scFv antibody phage library by the procedures described above and, as a result, a specific clone could successfully be obtained. This antibody specifically bound to not only S1T cells but also other ATL related cell lines but scarcely to ATL unrelated cell lines or non-activated T cells. From this, an antigen recognized by this antibody may possibly be an excellent cancer marker of ATL. Furthermore, the obtained antibody (a dimer of scFv), as inducing apoptosis in S1T cells, is expected for use as a remedy to cancer cells.

The amino acid sequences of VH chain and VL chain of scFv having the binding activity as described above and the nucleotide sequences coding for the same are as described below. The amino acid sequence of VH chain of the clone MS-S1TA3 is shown in SEQ ID NO: 1. Also, the amino acid sequences of CDR1 to CDR3 of said VH chain are shown in SEQ ID NOs: 2 to 4, respectively. Thus, in the amino acid sequence of VH chain as depicted in SEQ ID NO: 1, the amino acid sequence of the amino acid residues No. 31 to No. 35 corresponds to CDR1 (SEQ ID NO: 2), the amino acid sequence of the amino acid residues No. 50 to No. 66 to CDR2 (SEQ ID NO: 3), and the amino acid sequence of the amino acid residues No. 99 to No. 111 to CDR3 (SEQ ID NO: 4). The nucleotide sequence coding for the amino acid sequence of said VH chain is shown in SEQ ID NO: 5.

The amino acid sequence of VL chain of the clone MS-S1TA3 is shown in SEQ ID NO: 6. Also, the amino acid sequences of CDR1 to CDR3 of said VL chain are shown in SEQ ID NOs: 7 to 9, respectively. Thus, in the amino acid sequence of VL chain as depicted in SEQ ID NO: 6, the amino acid sequence of the amino acid residues No. 23 to No. 33 corresponds to CDR1 (SEQ ID NO: 7), the amino acid sequence of the amino acid residues No. 49 to No. 55 to CDR2 (SEQ ID NO: 8), and the amino acid sequence of the amino acid residues No. 88 to No. 98 to CDR3 (SEQ ID NO: 9). The nucleotide sequence coding for the amino acid sequence of said VL chain is shown in SEQ ID NO: 10.

The antibody and a fragment thereof of the present invention may encompass not only those with VH chain and VL chain and CDRs having the amino acid sequences as depicted in the respective SEQ ID NOs but also mutated polypeptides having said amino acid sequences with partial modification thereof. Thus, the antibody and a fragment thereof of the present invention includes polypeptides that have any of the amino acid sequences as depicted in the respective SEQ ID NOs with one or more amino acid residues therein being substituted, deleted, inserted and/or added and that may serve as a complementarity determining region of an H chain or an L chain of an antibody that recognizes HLA-DR β chain and has an apoptosis-inducing ability in cells expressing said HLA-DR β chain. One or more amino acid residues to be substituted, deleted, inserted and/or added may include, for instance, 1 to 12 amino acid residues, preferably 1 to 6 amino acid residues, and more preferably 1 to 3 amino acid residues.

A technique for introducing mutation into an amino acid sequence of a protein to obtain a functionally equivalent protein is known in the art. For instance, a technique for introducing mutation includes site specific mutagenesis (Current Protocols in Molecular Biology edit. 1987, 5: Section 8, 1-8), random introduction of mutation in an antibody variable region (PCR methods and Applications, 1992, 2: 28-33) and an approach where antibody affinity is enhanced in vitro (Nature Medicine, 1996, 2: 100-102). From the antibodies obtained by these techniques may be selected an anti-HLA-DR β chain antibody endowed with the property of the present invention.

Such “mutation” chiefly refers to ones artificially introduced by the techniques for preparing mutated proteins known in the art but may also be obtained as a consequence of isolation and purification of naturally occurring, e.g. in human, polypeptides likewise mutated.

In another preferable embodiment, VH chain of the anti-HLA-DR β chain antibody of the present invention has an amino acid sequence with 90% or more homology to the amino acid sequence as depicted in SEQ ID NO: 1.

In another preferable embodiment, VL chain of the anti-HLA-DR β chain antibody of the present invention has an amino acid sequence with 90% or more homology to the amino acid sequence as depicted in SEQ ID NO: 6.

Besides, in another preferable embodiment, the anti-HLA-DR β chain antibody of the present invention has such an amino acid sequence that:

(a) VH chain of said antibody has an amino acid sequence with 90% or more homology to the amino acid sequence as depicted in SEQ ID NO: 1; and

(b) VL chain of said antibody has an amino acid sequence with 90% or more homology to the amino acid sequence as depicted in SEQ ID NO: 6.

An extent of an amino acid sequence homology may be, for instance, about 90%, more preferably about 95%, most preferably about 97%, about 98% or about 99% or more. Homology of an amino acid sequence may be determined by the methods known in the art. For instance, homology (%) of an amino acid sequence may be determined by employing program commonly used in the art (e.g. BLAST, FASTA, and the like) with initialization. In another aspect, homology (%) may be determined by employing algorithm known in the art, e.g. algorithm of Needleman et al. (J. Mol. BioR1970, 48: 444-453), Myers and Miller (CABIOS, 1988, 4: 11-17), and the like. The algorithm of Needleman et al. is integrated into GCG software package (available from www.gcg.com) and homology (%) may be determined by employing e.g. BLOSUM 62 matrix or PAM250 matrix, and gap weight: 16, 14, 12, 10, 8, 6 or 4, and length weight: 1, 2, 3, 4, 5 or 6. The algorithm of Myers and Miller is integrated into ALLIGN program which is a part of GCG sequence alignment software package. When ALIGN program is used for comparing amino acid sequences, e.g. PAM120 weight residue table, gap length penalty 12, gap penalty 4 may be employed. Amino acid sequence homology may be one determined by any of the methods as described above but may be one determined by such a method that shows the lowest value for calculation.

The VH chain and/or VL chain as disclosed by the present invention, though being obtained in the form of scFv with phage display technique and being estimated in the form of scFv or diabody, may not be limited to these molecular species in principle. For instance, the present invention may also encompass other antibody fragments, including a whole antibody comprising the disclosed VH chain and/or VL chain bound to a constant region of a human immunoglobulin, or Fab, Fab′ or F(ab′)₂ comprising the disclosed VH chain and/or VL chain bound to a portion of a constant region of a human immunoglobulin, or a single chain antibody (scAb) comprising scFv bound to a constant region of an L chain of a human immunoglobulin, or (scFv)₂ which is prepared by linking two scFv to form a single chain molecule, and the like.

Alternatively, the antibody or a fragment thereof of the present invention may be fused with peptides or other proteins to form a fused antibody.

Besides, the present invention may also encompass a modified protein molecule wherein these antibodies or a fragment thereof are bound with a high molecular weight modifying agent such as polyethylene glycol.

For preparing scFv wherein an H chain and an L chain are linked together via a linker, a peptide linker, e.g. any single chain peptide comprising 10 to 25 amino acid residues, may be used. In this regard, it is known that a shortened length of a linker such as e.g. of about 5 amino acid residues will facilitate dimer formation to form a diabody.

The antibody or a fragment thereof of the present invention may be expressed in a suitable host, e.g. bacteria, or yeasts, by introducing genes coding for VH chain and VL chain of the respective clones obtained in accordance with the present invention as depicted in SEQ ID NOs: 5 and 10.

The antibody or a fragment of the antibody or a derivative thereof of the present invention may be used as a detection reagent or a diagnostic for various cancers such as ATL. Furthermore, the antibody or a fragment of the antibody or a derivative thereof of the present invention may be used as a medicament for treating various cancers such as ATL.

The present invention is explained in more detail by means of the following Examples but should not be construed to be limited thereto.

### EXAMPLE 1

### Construction of Phage Library from Healthy Donors

Referring to J. D. Marks et al., J. Mol. Biol., 222: 581-597, 1991, phage library was constructed using as a starting material lymphocytes from peripheral blood taken from 20 healthy donors. It was estimated that each of the constructed sublibraries VH(γ)-Vκ, VH(γ)-Vλ, VH(μ)-Vκ, and VH(μ)-Vλ exhibited diversity of 1.1×10⁸, 2.1×10⁸, 8.4×10⁷ and 5.3×10⁷ clones, respectively.

### EXAMPLE 2

### Screening with ATL Cell Line

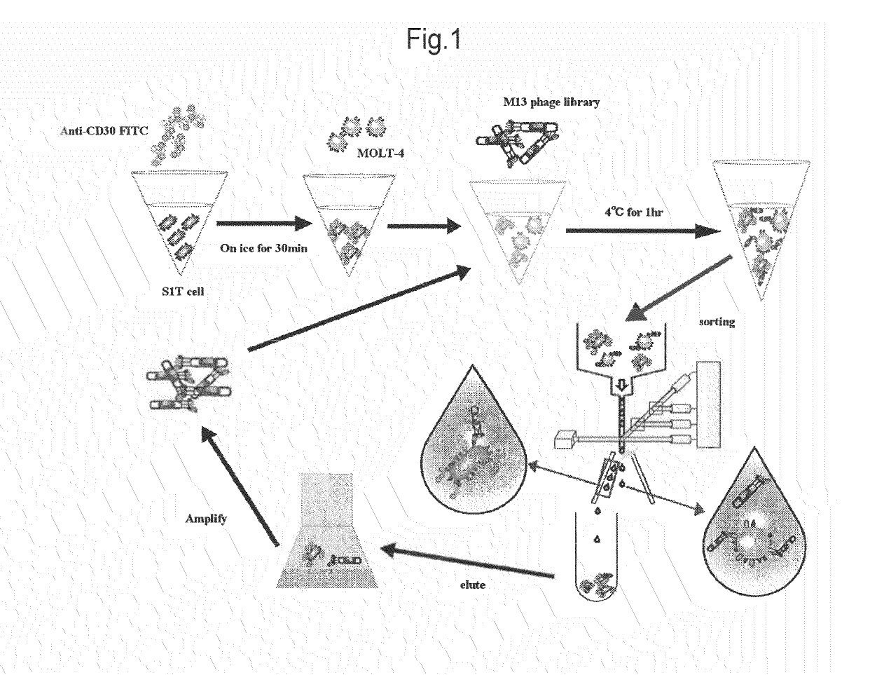

For preparing an antibody specific to S1T cells, ATL cell line, a panning process with a flow cytometer was devised as shown in FIG. 1. Thus, to a mixture (500 μl, 1% BSA in PBS) of 10⁶ MOLT4 cells and an equivalent amount of S1T cells previously labeled with anti-CD30 antibody bound with FITC was added the antibody phage library at 10¹² TU and the mixture was reacted at 4° C. for 1 hour while stirring slowly. After centrifugation (3,000×30 sec.), supernatant was removed and precipitated cells were suspended in 500 μl of 1% BSA. In a similar manner, the cells were again washed and suspended in 8 ml of FACS buffer (0.1% NaN₃, 2% FBS in PBS). After filtration with a filter, about 2×10⁵ cells were separated with a flow cytometer. The separated cells were suspended in RPMI medium and, after centrifugation at 3,000 rpm for 6 min., the cells were collected. To the collected cells were added 100 μl of PBS (200 μl in total) and then 300 μl of 76 mM citric acid at pH 2.5. After the mixture was left to stand for 5 min., 500 μl of 1 M Tris-HCl (pH 7.4) was added for neutralization. The mixture was then transferred to 30 ml of culture of E. coli (TG1) and left to stand at 37° C. for 30 min. for infection. After culture for 30 min. and exchange of culture medium, the antibody phages were rescued in accordance with protocol. The collected antibody phages were again subject to the same panning processes. Panning processes were performed three times in total.

### EXAMPLE 3

### Analysis of Reactivity of Phage Antibody with Flow Cytometer

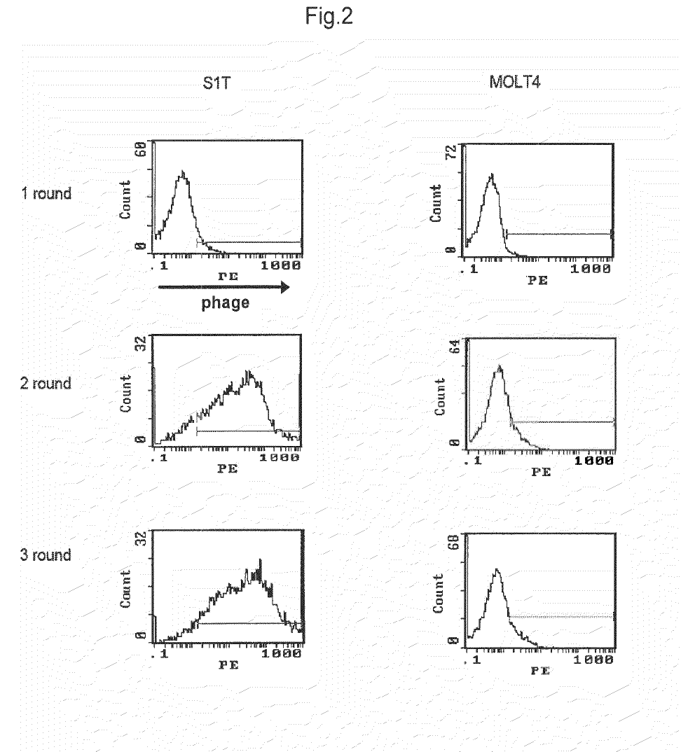

The solutions of antibody phages obtained in each of the panning processes were studied for their binding activity to S1T cells with a flow cytometer. The results are shown in FIG. 2. As shown in FIG. 2, after the 1st panning process, no binding activity was observed to both S1T cells and MOLT-4 cells but the antibody phages obtained after the 2nd and 3rd panning processes exhibited a shift of fluorescent intensity in peaks apparently attributable to binding activity only for S1T cells. From this, it was found that antibody phages specifically binding to S1T cells were concentrated.

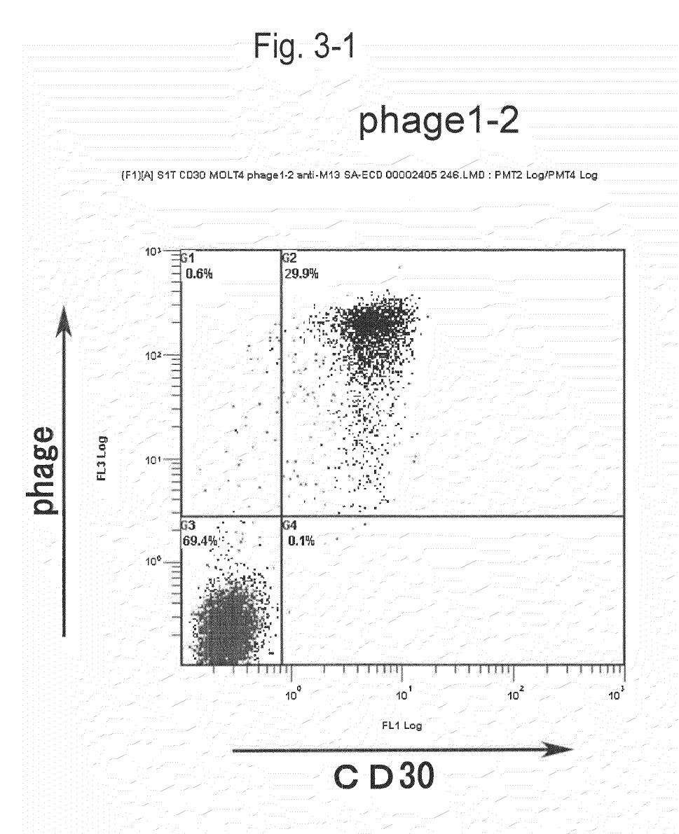

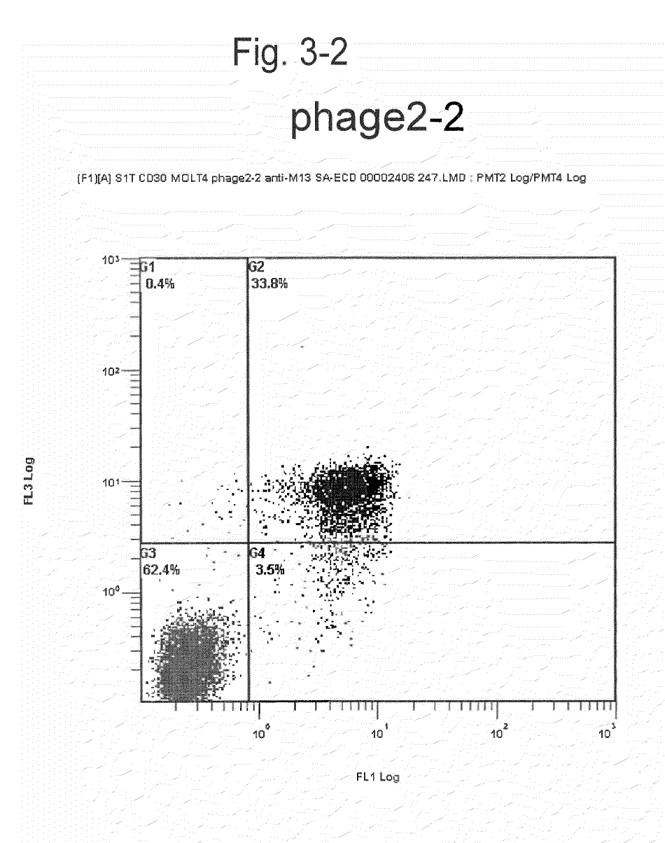

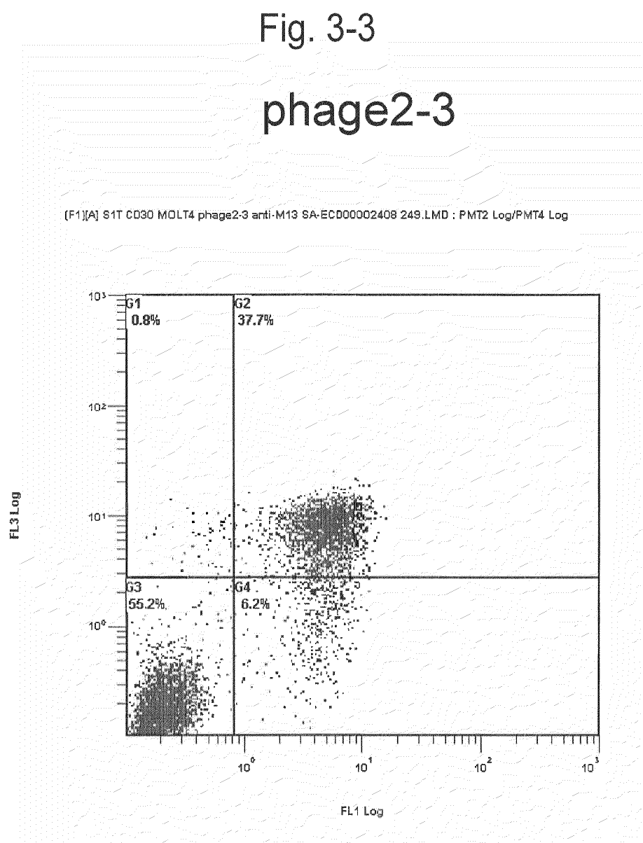

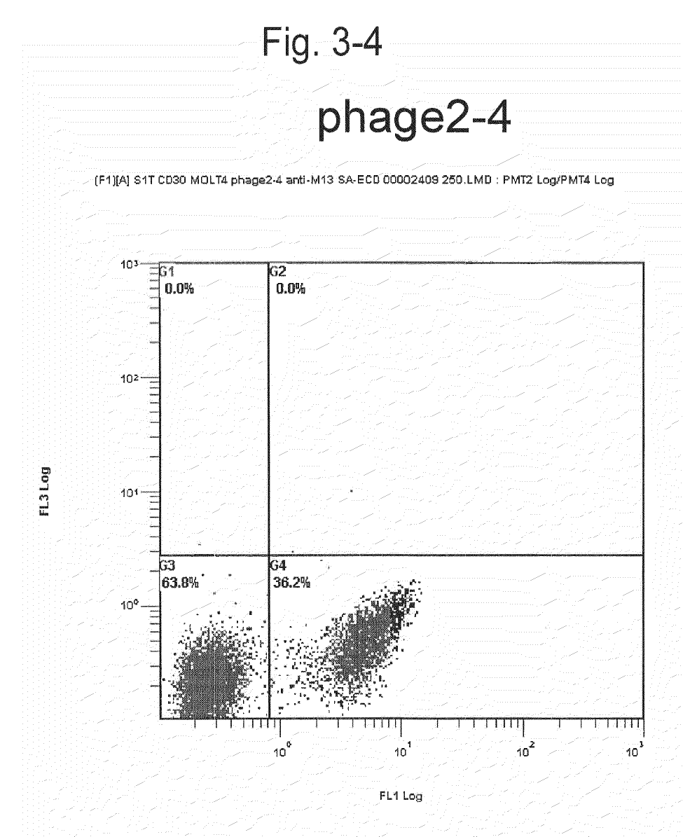

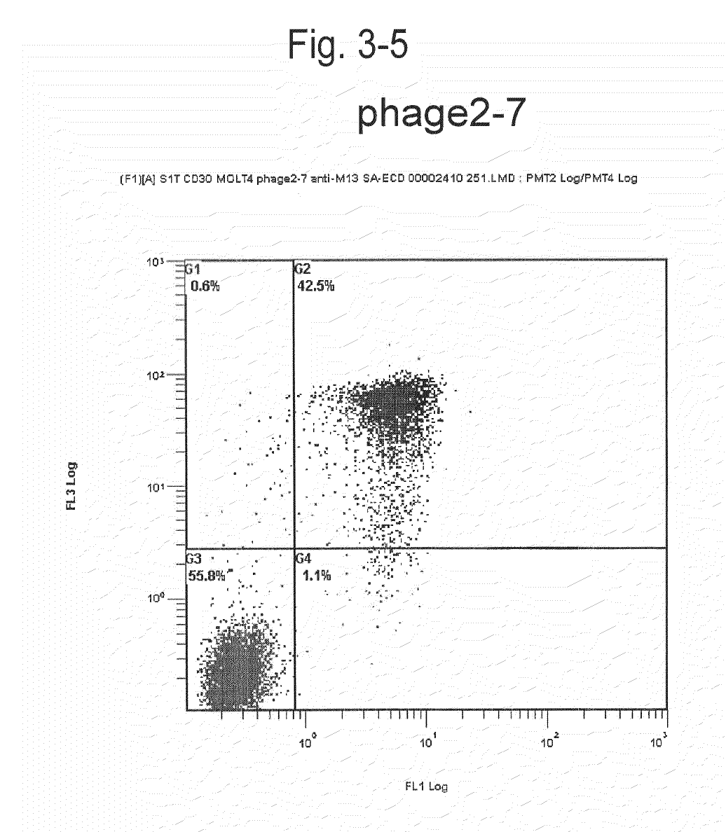

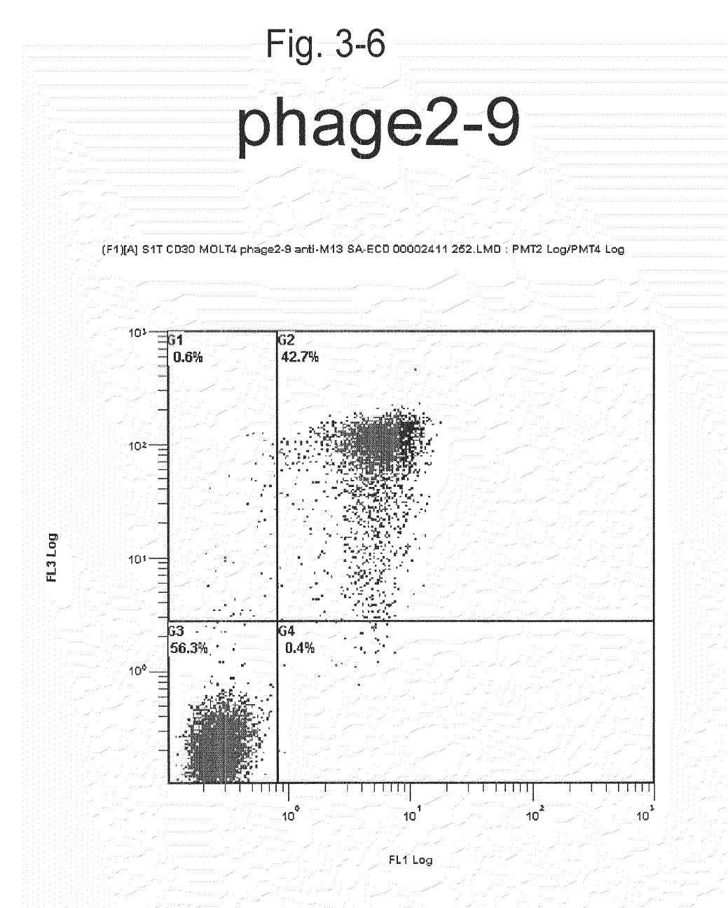

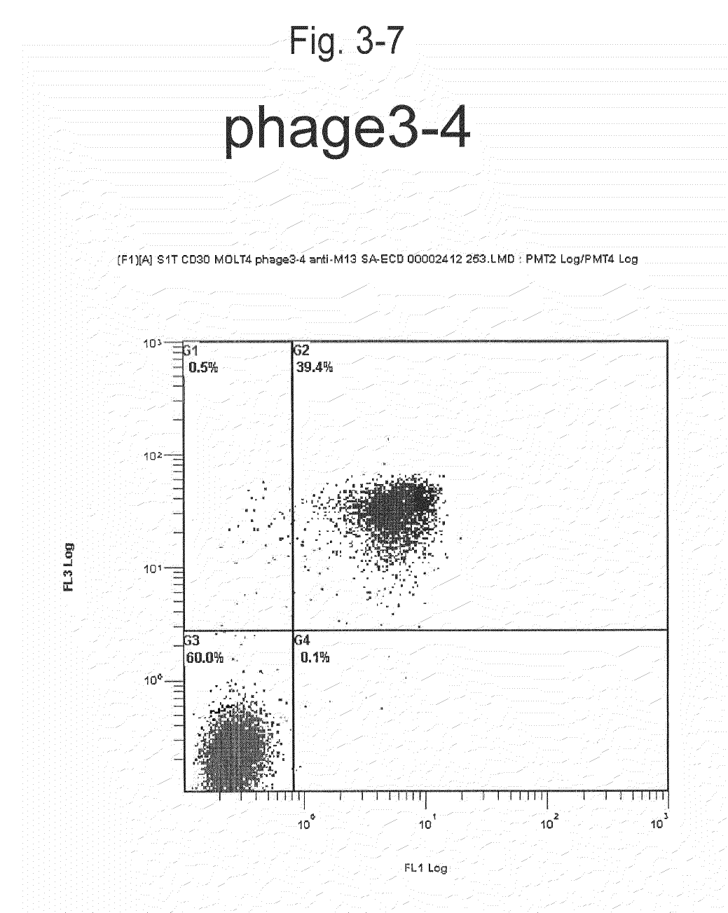

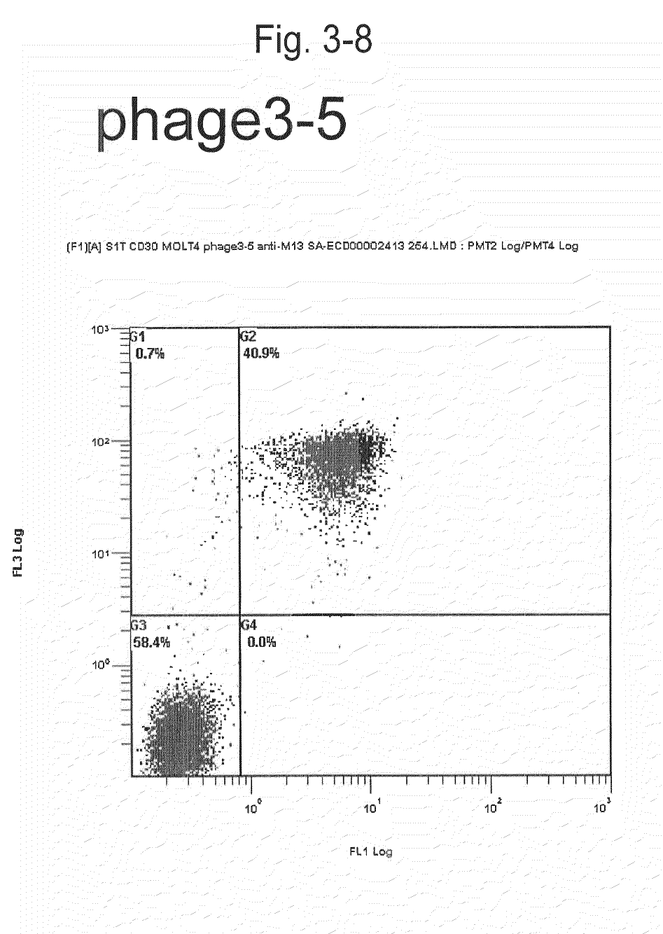

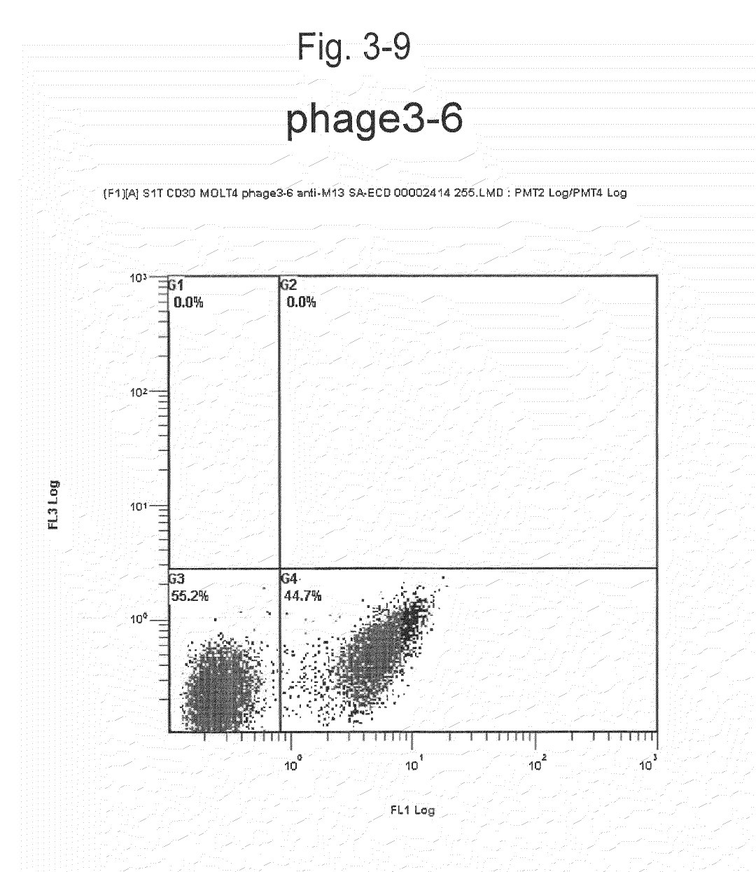

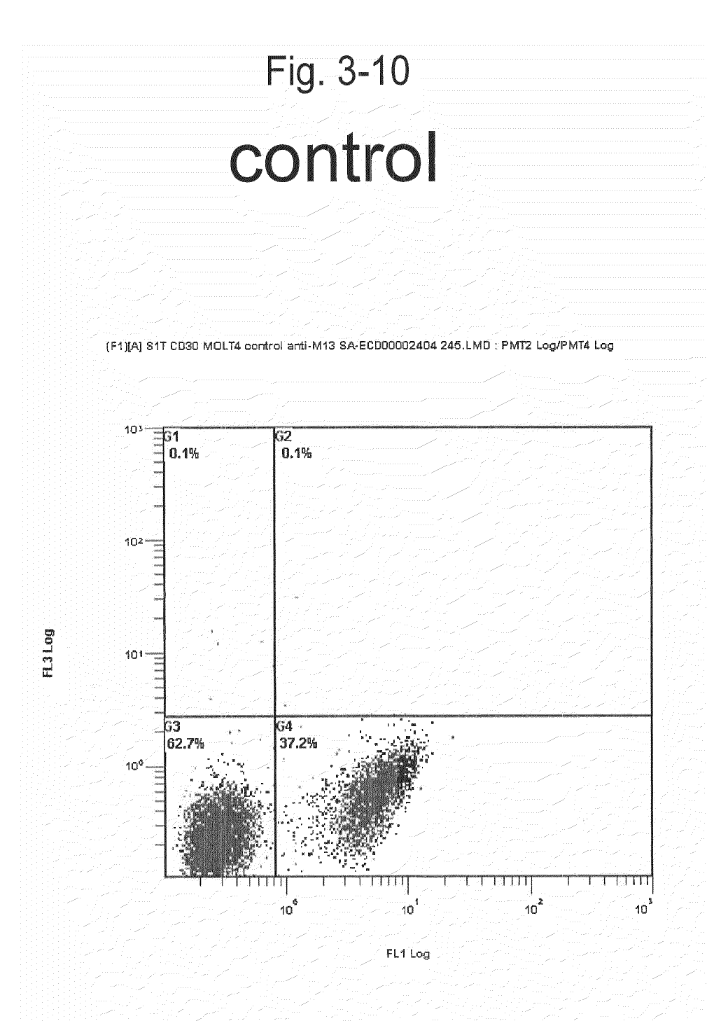

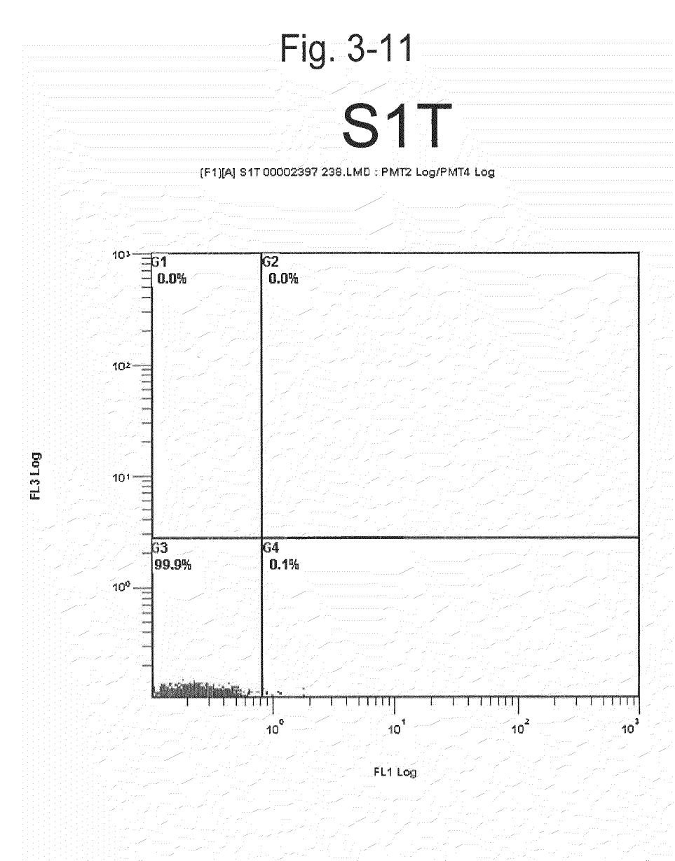

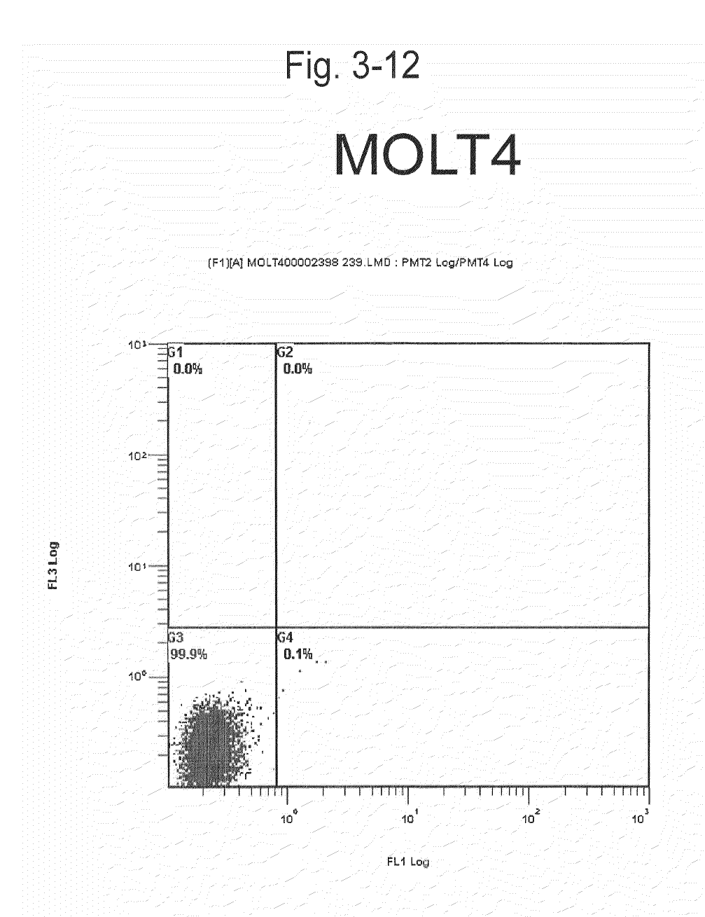

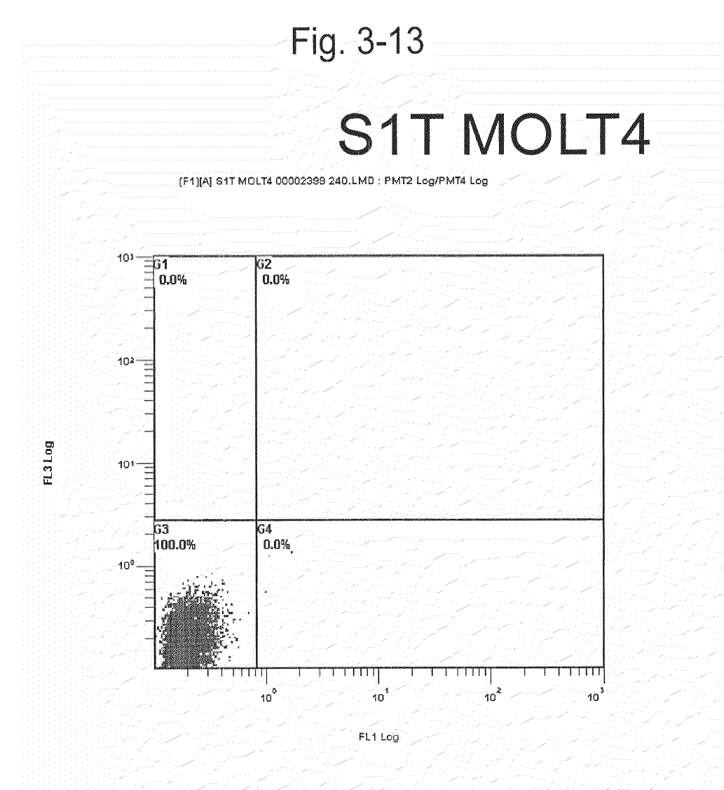

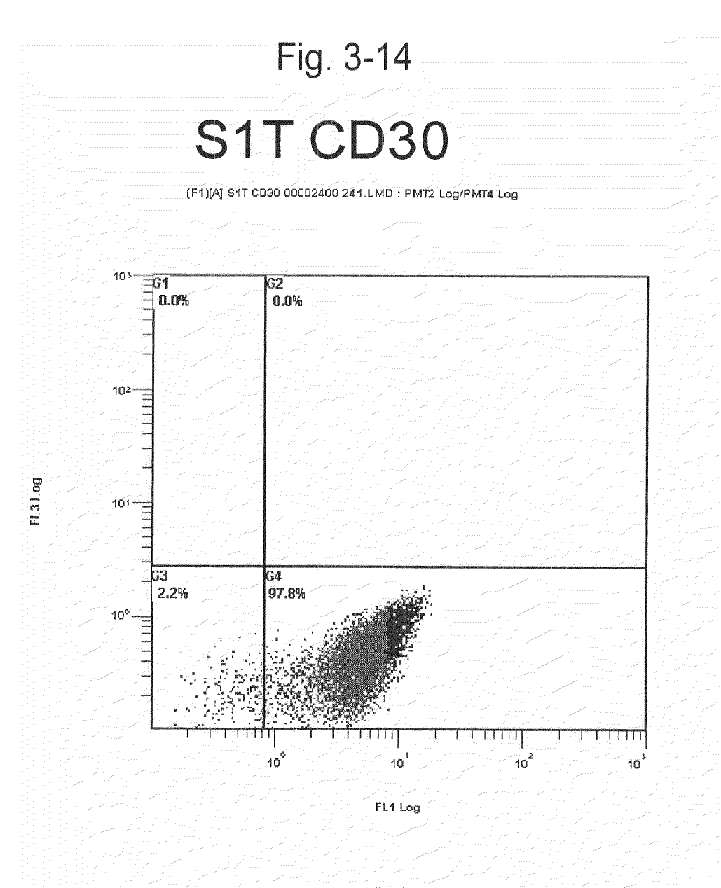

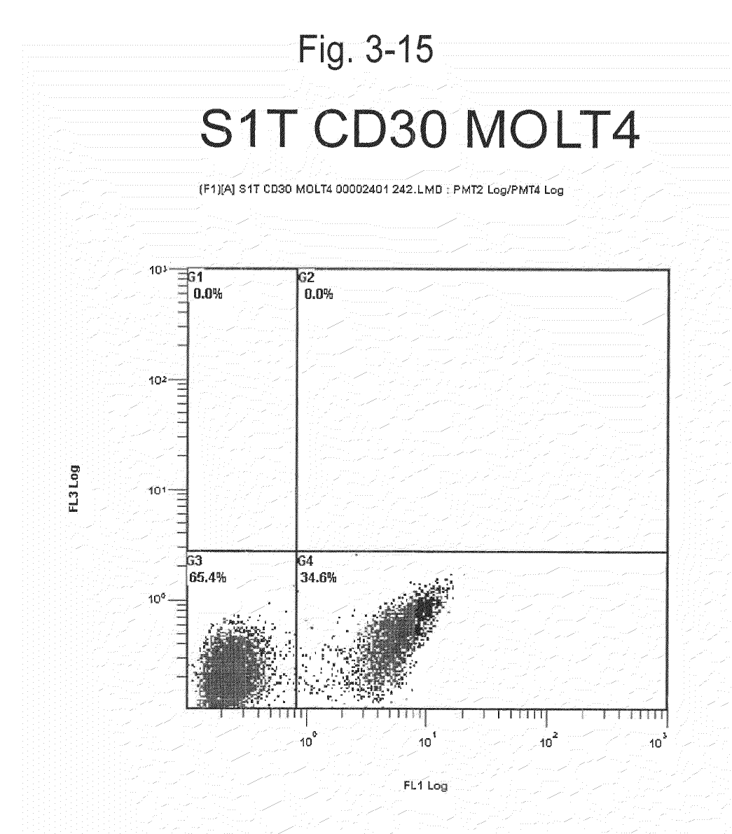

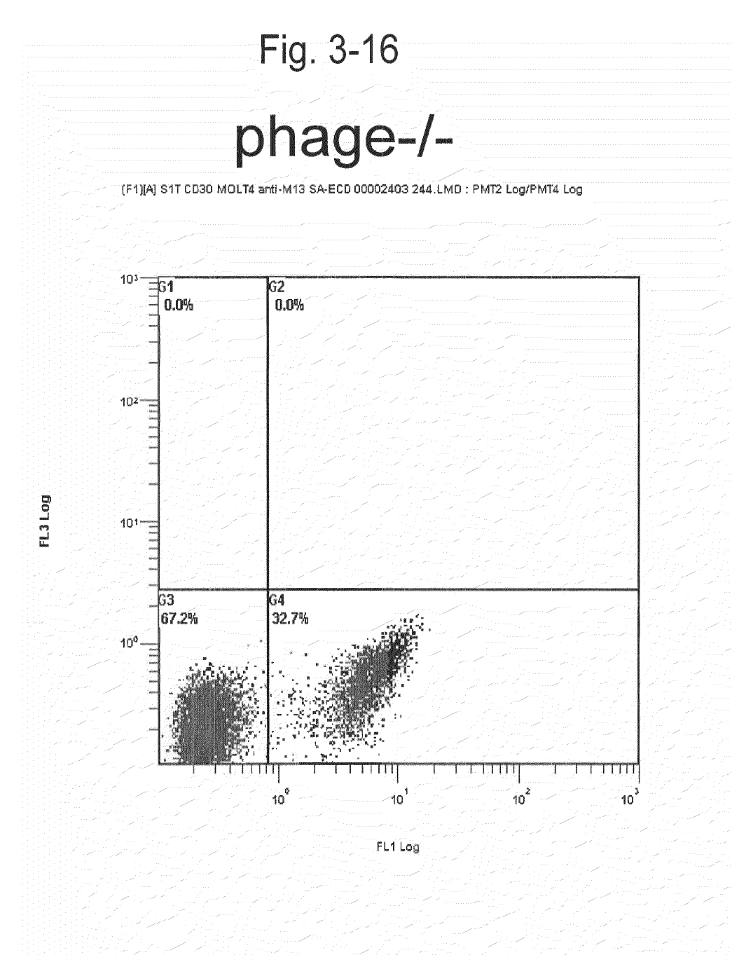

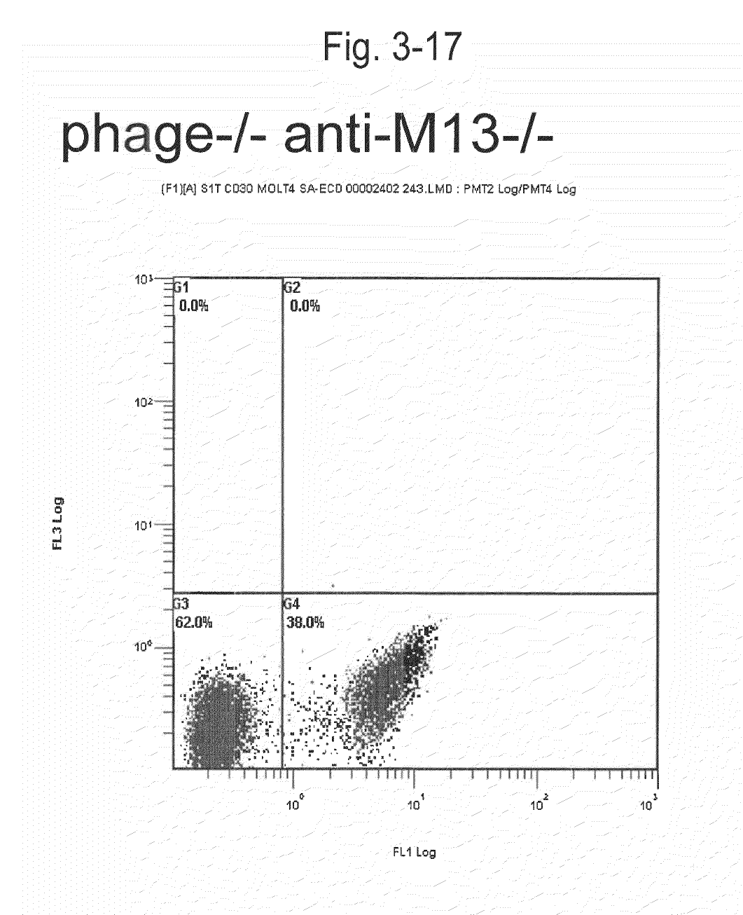

Thus, the antibody phages obtained after the 2nd and 3rd panning processes were infected to E. coli and, after cloning, the binding activity of each of phage clones to S1T cells were analyzed with a flow cytometer. As a result, 5 clones among 14 clones after the 2nd panning process and 2 clones among 14 clones after the 3rd panning process showed binding activity (FIG. 3).

### EXAMPLE 4

### Sequencing of Specific Clones

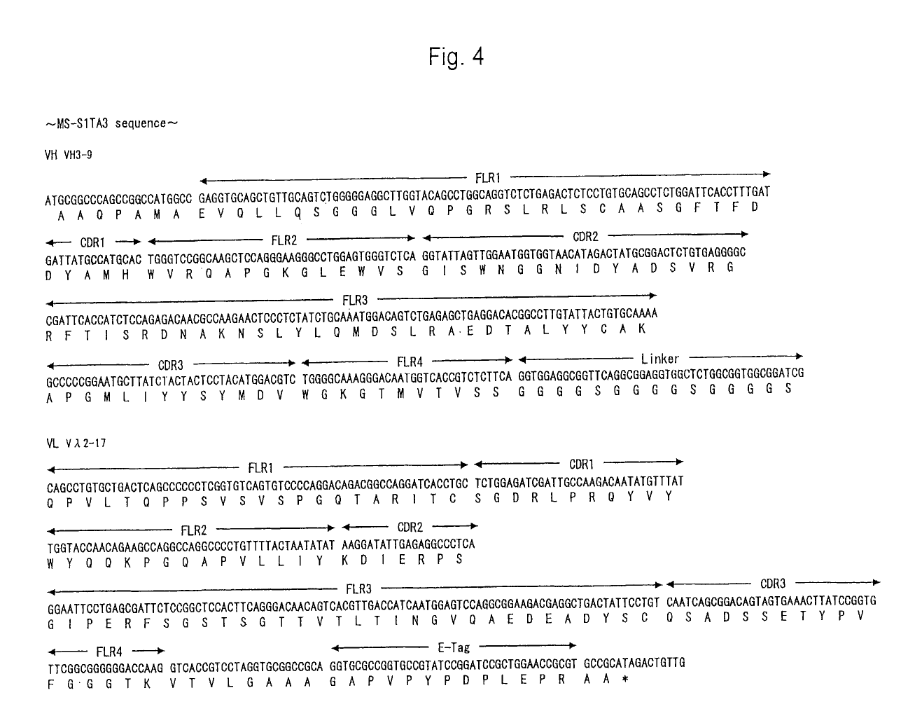

The clones were subject to DNA sequencing to reveal that all of the clones had one and the same sequence. FIG. 4 shows amino acid sequences translated from the DNA sequences of VH and VL regions of an antibody.

### EXAMPLE 5

### Expression and Purification of scFv

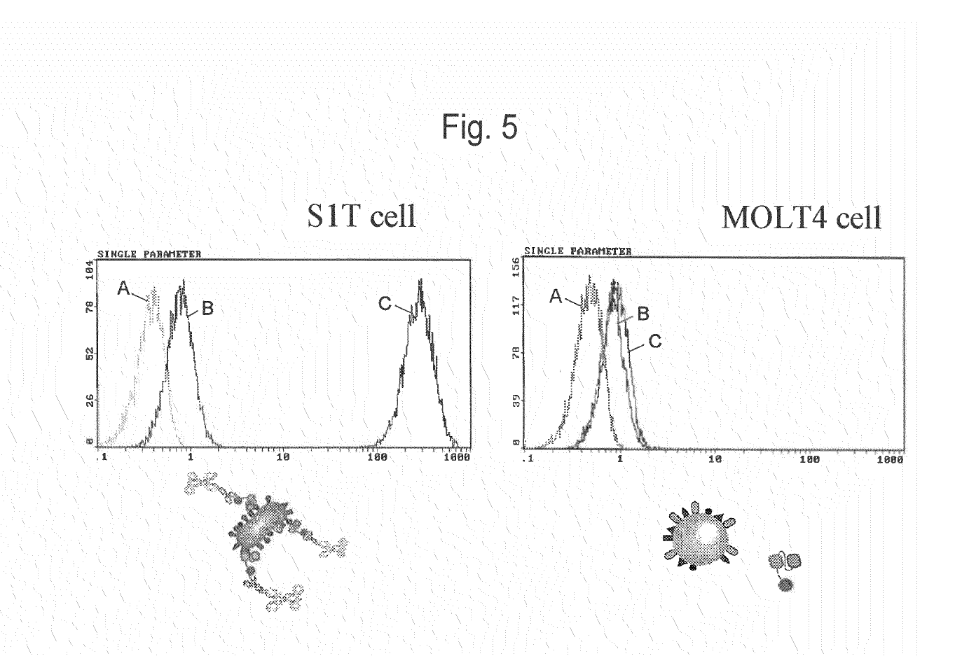

The obtained scFv, when expressed in E. coli, were localized in the cytoplasm rather than in the periplasm fractions. For facilitating purification of scFv, the E-tag at the C-terminus for detection and purification was replaced with His tag and scFv was produced in E. coli HB2151 by IPTG induction. scFv purified with His tag column was tested for its binding to S1T cells. The results are shown in FIG. 5.

### EXAMPLE 6

### Analysis of Reactivity of scFv with Flow Cytometer

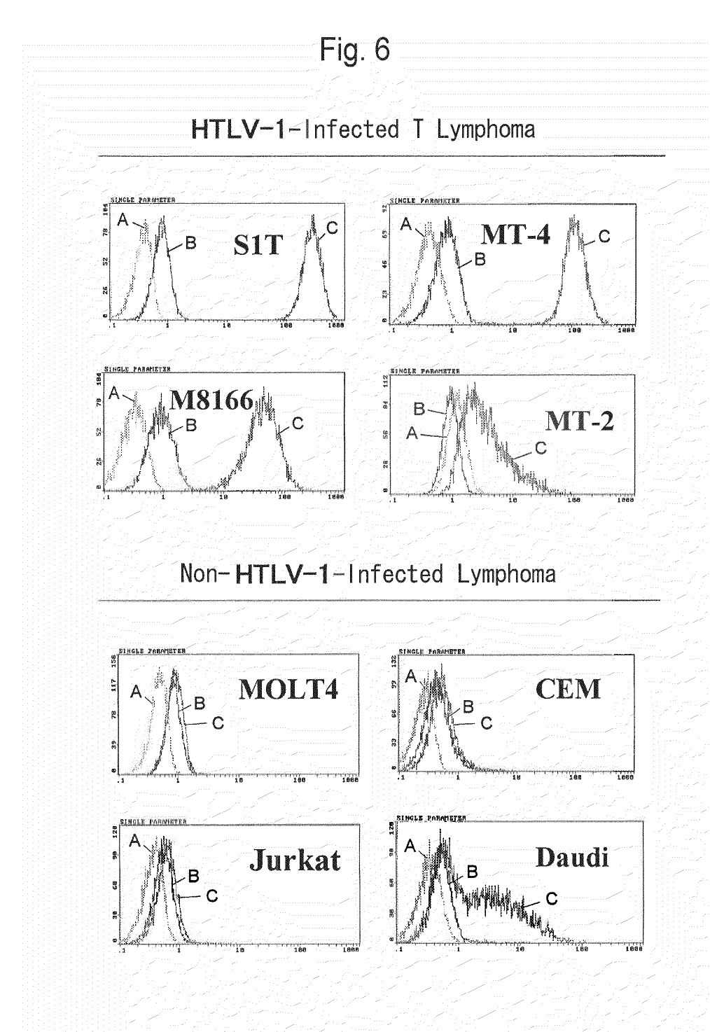

The binding activity of the obtained scFv antibody (MS-S1TA3) was compared between various HTLV-1-infected cell lines and non-infected cell lines. The results are shown in FIG. 6. The antibody bound to the HTLV-1-infected T cell lines, S1T, MT-4, and M8166, comparatively strongly and also to MT-2 though weakly. On the other hand, binding was scarcely observed for the non-infected cell lines from T cells but was observed for Daudi cells which are B cell lymphoma. This suggests that the scFv antibody of the present invention may possibly be a specific antibody efficacious to HTLV-1-infected T cell lymphoma.

### EXAMPLE 7

### Identification of Antigen by LC-MS/MS Analysis

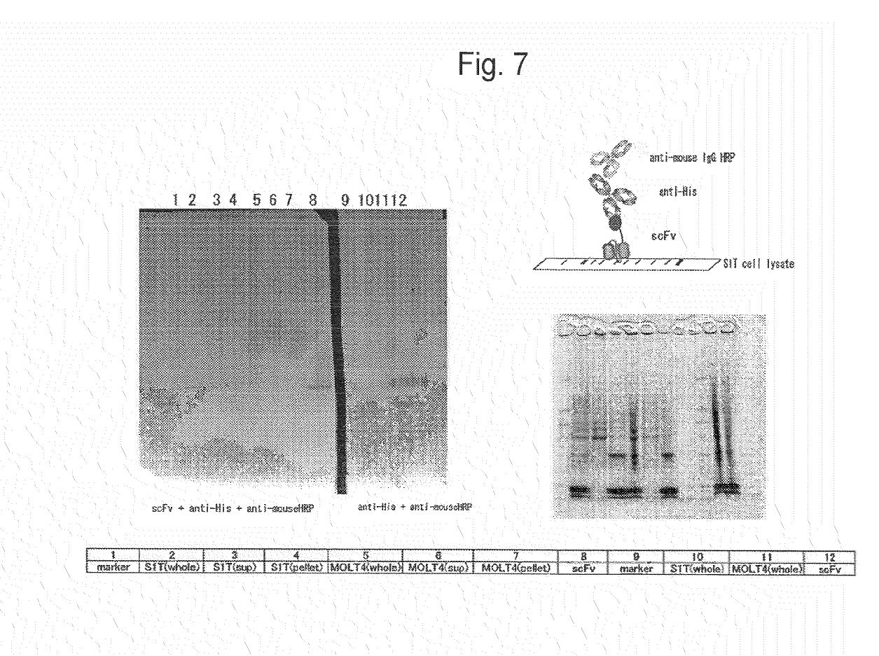

Next, for identifying an antigen for scFv antibody (MS-S1TA3), a lysate of S1T cells was subject to SDS-PAGE (under reduced condition) by Western blotting followed by immunostaining with scFv antibody. As a result, as shown in FIG. 7, a band was detected at M.W. of about 32,000, though weak. The band was cut from the gel after electrophoresis, reductive alkylation and trypsin digestion were carried out in the gel and the obtained peptide fragments were analyzed by LC-MS/MS analysis. From the obtained mass spectrum data of fragments, candidate proteins contained in the gel were narrowed down by MASCOT retrieval. As a result, among the top 50 proteins with high score, the protein solely identified on cell surface was HLA class II DR β chain.

### EXAMPLE 8

### Estimation of Reactivity with HLA-DR

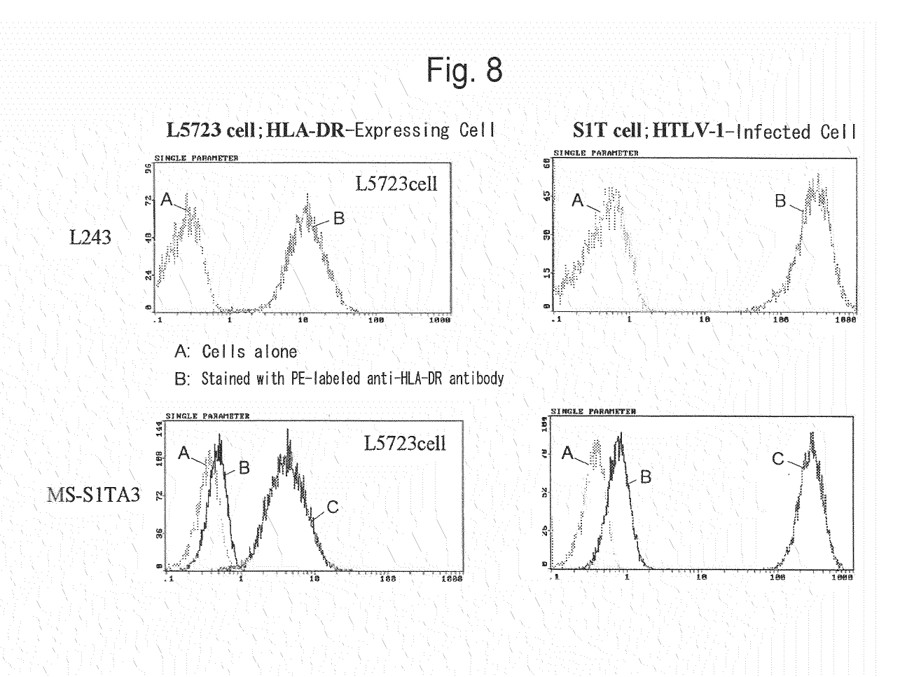

For ascertaining the results, binding property of scFv antibody (MS-S1TA3) to transformant L57.23, which is L cell transfected with genes encoding HLA-DR β chain (DRB1*0101) and HLA-DR α chain (DRA*0101), was surveyed with a flow cytometer. As a result, as shown in FIG. 8, binding property of reactivity similar to anti-HLA-DR α mouse antibody was observed.

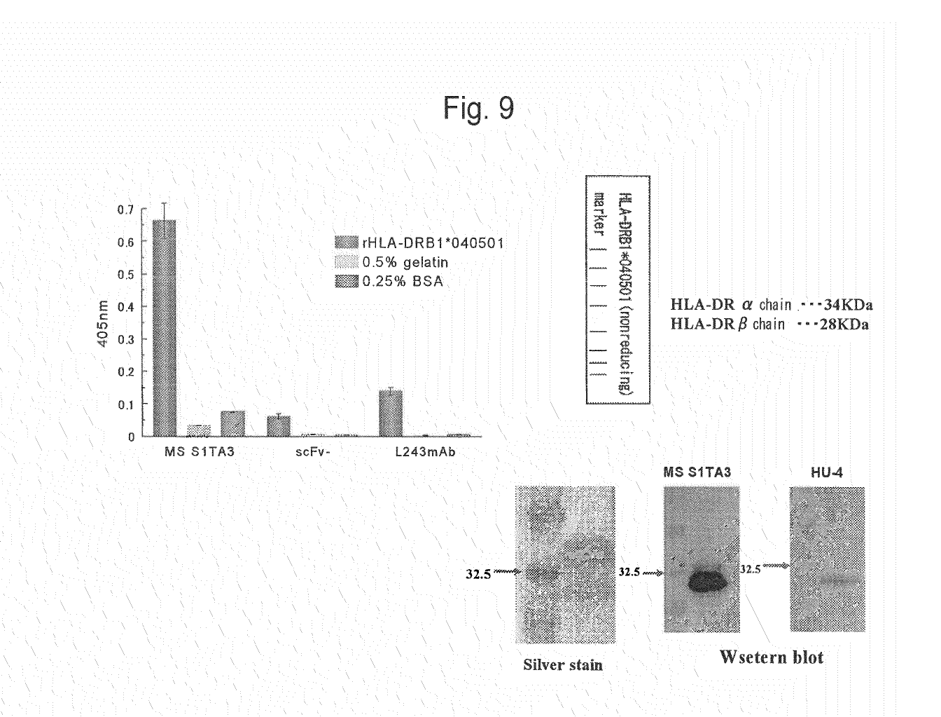

Furthermore, binding property of scFv antibody (MS-S1TA3) was surveyed using HLA-DR molecule (by courtesy of Sho Matsushita, Saitama Medical University) purified from human B cell strain EBwa of EB transform with anti-HLA antibody (FIG. 9). Thus, the binding property to HLA-DR protein derived from DRB1*0405 was estimated by ELISA to reveal apparent binding specificity of MS-S1TA3 antibody to HLA-DR as shown in FIG. 9, left panel. Besides, immunostaining by Western blotting indicated specific staining of the band of HLA-DR β chain at around 31 kDa but not the band of HLA-DR α chain at around 34 kDa. It was thus concluded that an antigen for scFv antibody (MS-S1TA3) was HLA-DR β chain.

However, in fact, the HLA gene includes multiple loci and alleles. HLA typing of S1T cells showed that HLA-DR β chain included HLA-DRB1*040501. Also, when the cDNA was amplified with HLA-DR β-specific primers and sequence analysis was performed, it was found that principal HLA-DR β chain molecule in S1T cells included not only HLA-DRB1 but also HLA-DRB4. From these, it was revealed that an antigen on S1T cells targeted by scFv antibody (MS-S1TA3) was either one or both of HLA-DR β chain proteins derived from these two genes.

### EXAMPLE 9

### Estimation of Ability of scFv to Induce Apoptosis

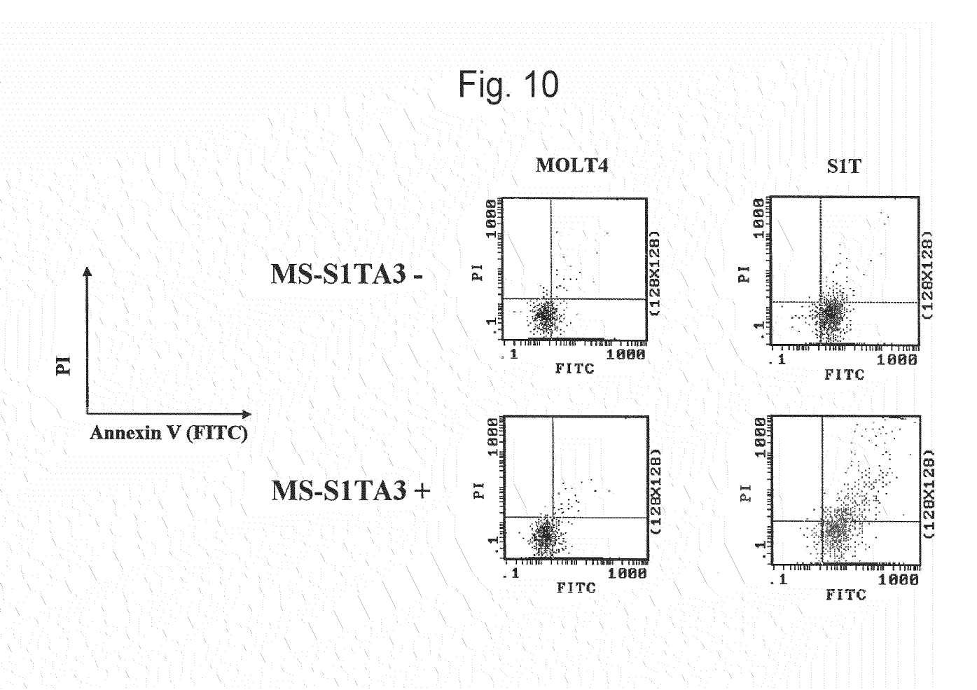

Quite interestingly, it was found that extremely potent apoptosis was induced by treating S1T cells with scFv antibody (MS-S1TA3) at 37° C. for 1 hour. About 50 μg/l of scFv antibody was added and, after 1 hour, the cells were stained with FITC-labeled Annexin V and PI and analyzed with a flow cytometer. The results are shown in FIG. 10. For the control MOLT-4, no difference was observed between addition and no addition of scFv antibody. For S1T cells, however, the addition of scFv antibody increased doubly positive cells to confirm the occurrence of apoptosis-like cell death.

### EXAMPLE 10

### Comparison of Activity in Various Molecular Formats

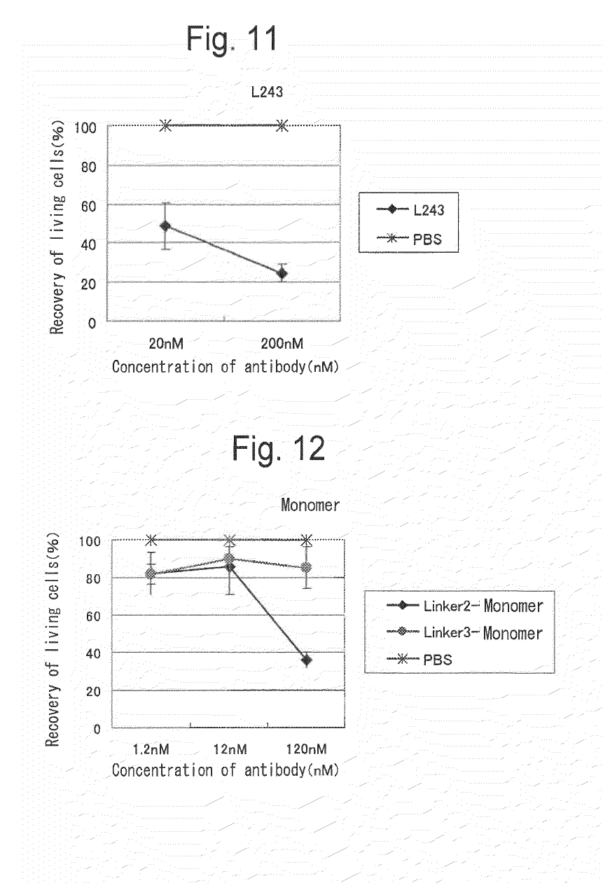

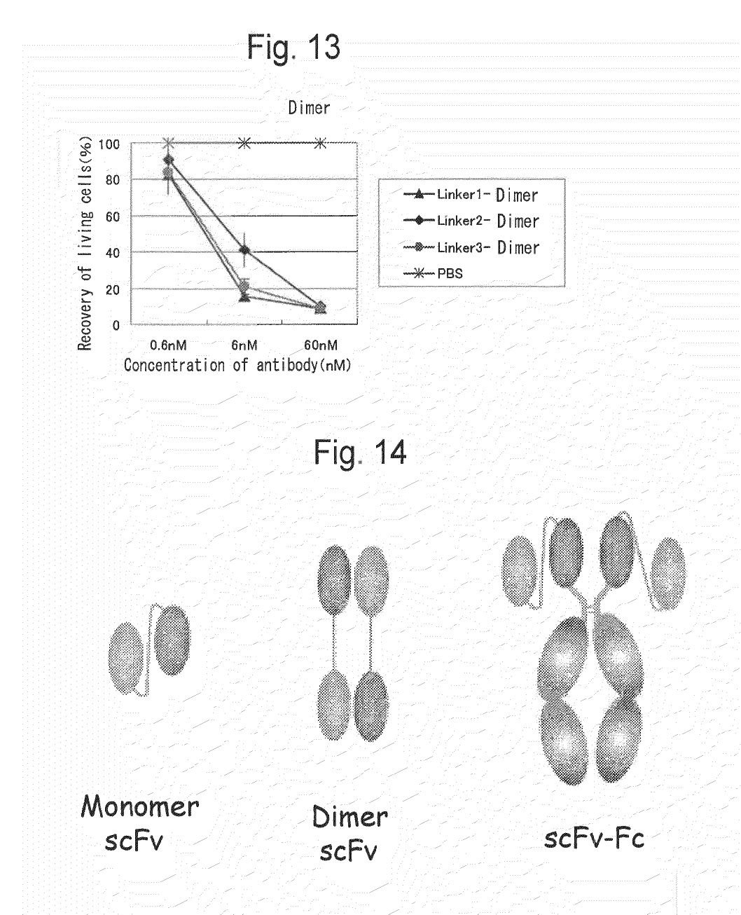

In general, it is reported that the format of scFv is such that a monomer and a dimer, i.e. a diabody, are present in admixture thereof. Thus, for investigating which format is a molecular species responsible for induction of apoptosis, a monomer and a dimer were separated from each other by molecular sieve chromatography and then compared for their apoptosis-inducing ability. Besides, from scFv where the linker is 15-mer in length (linker 3, (GGGGS)×3: SEQ ID NO: 15) were prepared scFv where the linker is 10-mer in length (linker 2, (GGGGS)×2: SEQ ID NO: 13) and scFv where the linker is 5-mer in length (linker 1, (GGGGS)×1; SEQ ID NO: 11) and a monomer and a dimer of the respective scFv were compared with each other for their apoptosis-inducing ability. Induction of apoptosis was measured by adding 1×10⁵ cells/100 μL and each of respective scFv to a 96-well plate for reaction at 37° C. for 2 hours in the presence of CO₂ and then counting cells with a flow cytometer (FIGS. 11 to 13). As a result, as shown in FIGS. 12 and 13, the monomer showed scarcely no or extremely weak induction of apoptosis whereas the dimer showed as much as 80% or more apoptosis at a concentration of 6 nM. The results suggest that apoptosis-like cell death induced by scFv antibody (MS-S1TA3) is such that HLA-DR β chain is transformed into a dimer with a dimer of scFv antibody to thereby induce a potent apoptosis. FIG. 11 compares apoptosis induction with L243 mouse monoclonal antibody to HLA-DR α chain which is reported to induce apoptosis. It deserves special attention that L243 induced apoptosis at EC₅₀ of 20 nM whereas a dimer of scFv antibody (MS-S1TA3) induced apoptosis at EC₅₀ of 1.5 nM, i.e. at about 10-fold lower concentration for the same effect as that of L243.

### EXAMPLE 11

### Production and Preparation of scFv-Fc

Since MS-S1TA3 antibody exhibited prominent apoptosis-inducing activity in the form of diabody whereas apoptosis induction by L243 antibody was not so potent, a possibility was conceived that a distance between the HLA-DR molecules cross-linked by an antibody might be important for induction of apoptosis. However, there remained a possibility that different epitopes recognized by MS-S1TA3 antibody and L243 antibody (MS-S1TA3 antibody recognizes HLA-DR β chain whereas L243 antibody recognizes HLA-DR α chain) might be the cause of difference in the apoptosis-inducing activity. Thus, scFv-Fc protein was prepared as a derivative of MS-S1TA3 antibody wherein Fc from human IgG1 was bound to scFv (FIG. 14) and its apoptosis-inducing activity was compared with that of a diabody of MS-S1TA3 antibody.

Genetic construction of scFv-Fc was carried out as described below. The gene of scFv of MS-S1TA3 antibody was amplified by PCR and inserted into a cloning site of a human Fc fusion protein expression vector. In this vector, a leader sequence for extracellular secretion, the scFv gene and the human Fc gene are linked together and its expression is regulated with CAG promoter. The vector also includes a neomycin resistant gene and a ampicillin resistant gene as a drug resistant gene.

scFv-Fc was expressed transiently with FreeStyle 293-F cell (Invitrogen) as a host. 293fectin (Invitrogen) was used for gene introduction and, after culture for 2 to 3 days on FreeStyle 293 expression medium (Invitrogen), culture supernatant was collected by centrifuge and filtration with a 0.22 μm filter.

Purification was carried out by ordinary procedures using Protein A column chromatography. A solution of scFv-Fc obtained after dialysis with PBS was used as a purified product.

### EXAMPLE 12

### Analysis of Reactivity of scFv-Fc

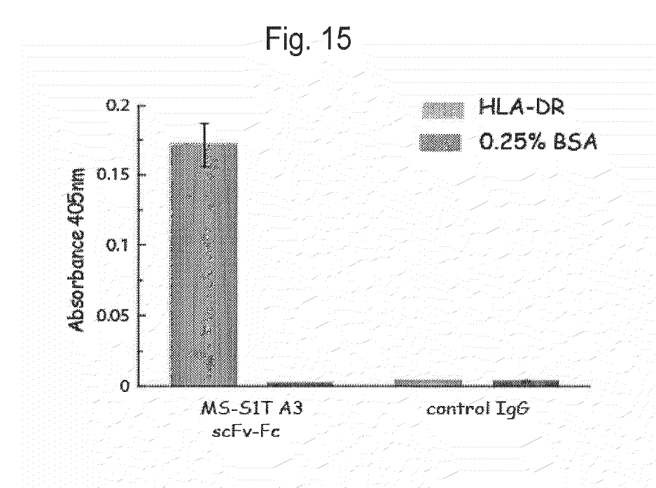

First, binding property of the produced MS-S1TA3 scFv-Fc to HLA-DR was estimated by ELISA. A plastic plate was coated with 50 ng of HLA-DR and blocked with 0.25% BSA. After the produced scFv-Fc (50 nM) was reacted, bound scFv-Fc was detected with AP-labeled anti-human Fc goat Fab antibody. The results are shown in FIG. 15. scFv-Fc specifically reacted with a well coated with HLA-DR to confirm that the produced scFv-Fc maintained binding property to HLA-DR.

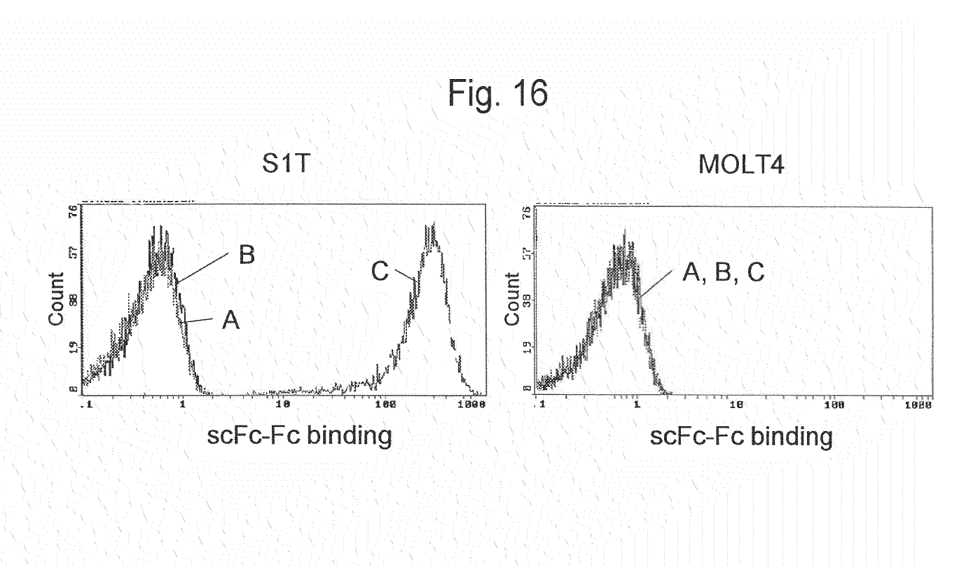

Next, binding property of the scFv-Fc was estimated with a flow cytometer. As a result, as shown in FIG. 16, scFv-Fc antibody of MS-S1TA3 bound to S1T cells quite intensively but never to the control cells MOLT4.

### EXAMPLE 13

### Comparison of Apoptosis Induction to S1T Cells between scFv-Fc and Diabody

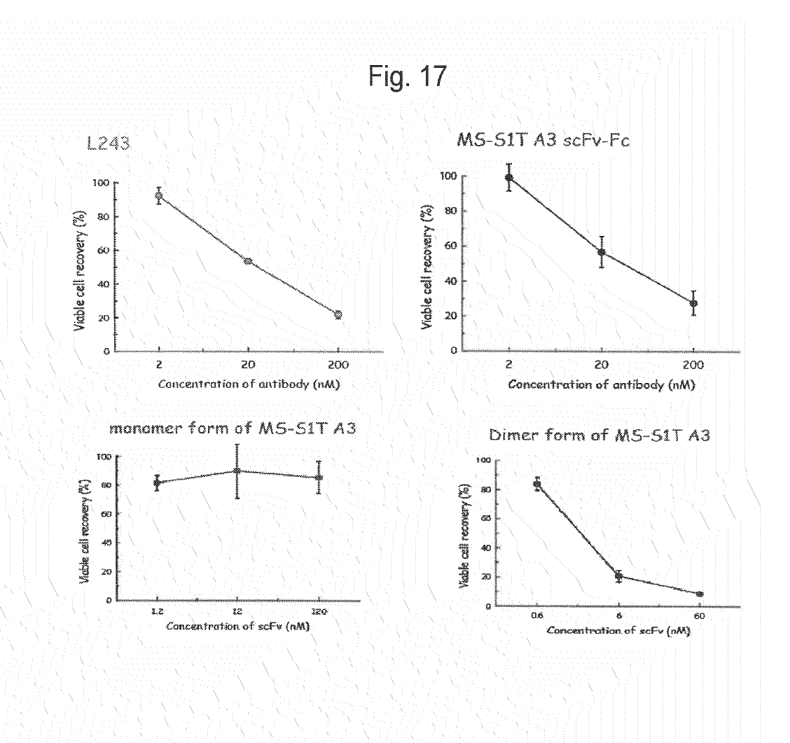

For the scFv-Fc where the binding specificity was confirmed, an apoptosis-inducing activity to S1T cells was estimated. The results are shown in FIG. 17. scFv-Fc induced apoptosis in 50% of the cells at about 30 nM, which activity was a level similar to that of L243 antibody. On the other hand, the diabody induced apoptosis in 50% of the cells at about 2 nM. It was thus found that the diabody had apparently a more potent apoptosis-inducing activity than that of scFv-Fc.

These results demonstrate that a shorter distance between the HLA-DR proteins to be cross-linked by an antibody might be significant for inducing potent apoptosis and strongly suggest that a dimer structure of scFv (diabody) might be more efficacious for induction of cell death signal via HLA-DR than an ordinary intact antibody or scFv-Fc antibody.

### EXAMPLE 14

### Estimation of Apoptosis Induction to Various Cells Expressing HLA-DR

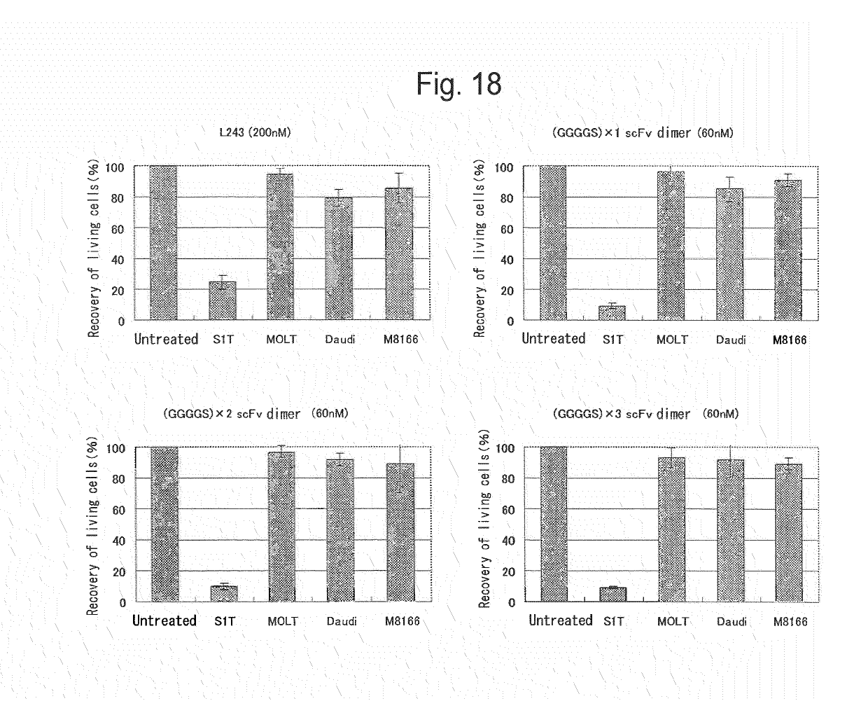

Whether apoptosis by a diabody of scFv antibody (MS-S1TA3) may also be induced in cells other than S1T cells was investigated. The results are shown in FIG. 18. Under the conditions where apoptosis is induced in 90% or more of S1T cells, no apoptosis was observed for MOLT-4 cells with any diabodies of scFv with different linkers whereas weak apoptosis was observed for Daudi cells or M8166 cells, i.e. in about 8 to 15% of the cells.

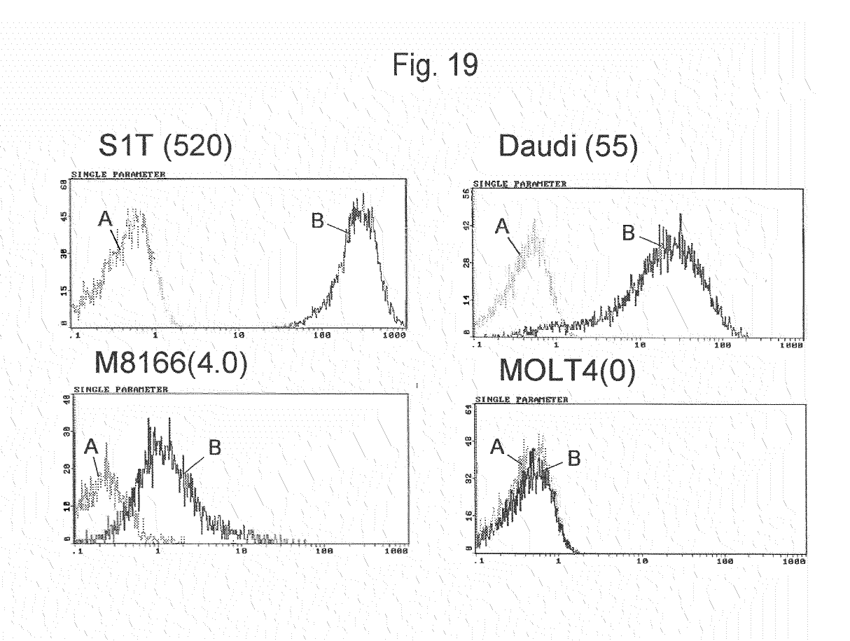

In order to investigate what is the cause of this difference in effects, an expression level of HLA-DR on the cells was determined by FACS analysis using an anti-HLA-DR α chain antibody (L243). As shown in FIG. 19, S1T cells exhibited intense fluorescence (520 of a median) by staining with the anti-HLA-DR α chain antibody whereas Daudi cells and M8166 cells showed fluorescence reduced to about 1/10 and 1/100 of that of S1T cells, respectively. These results were almost the same as those of cell staining with scFv antibody (MS-S1TA3) as in FIG. 6. From these, it was conceived that a lower expression level of HLA-DR in Daudi and M8166 cells as compared to S1T cells might be the cause of lower apoptosis induction of scFv antibody (MS-S1TA3) as shown in FIG. 18.

### INDUSTRIAL APPLICABILITY

In view of the results described above, the antibody and a fragment of said antibody of the present invention, which is a human antibody that may target HLA-DR β chain, the cell surface molecule highly expressed in adult T cell leukemia (ATL), and induce apoptosis in ATL cells, may greatly be expected for use in early diagnosis and therapy of a variety of cancers including ATL.

1-29. (canceled)

30. A human anti-HLA-DR β chain antibody fragment which is a diabody comprising an H chain variable region consisting of a polypeptide consisting of the amino acid sequence as depicted in SEQ ID NO: 1 and an L chain variable region consisting of a polypeptide consisting of the amino acid sequence as depicted in SEQ ID NO: 6.

31. The human anti-HLA-DR β chain antibody fragment according to claim 30 wherein said antibody fragment is a fusion of said diabody with Fc.

32. A fused antibody comprising the antibody fragment according to claim 30 fused to a peptide or other protein.

33. A modified antibody which is formed by binding to a modifying agent the antibody fragment according to claim 30 or the fused antibody comprising the antibody fragment according to claim 30 fused to a peptide or other protein.

34. A gene coding for the antibody fragment according to claim 30 or the fused antibody comprising the antibody fragment according to claim 30 fused to a peptide or other protein.

35. A recombinant expression vector comprising the gene according to claim 34.

36. A transfectant with the gene according to claim 34 introduced therein.

37. A method for producing a human anti-HLA-DR β chain antibody fragment by expressing the gene according to claim 34 in a host cell.

38. A reagent for detection and/or a diagnosis of cancers and/or viral infectious diseases comprising the antibody fragment according to claim 30, the fused antibody comprising the antibody fragment according to claim 30 fused to a peptide or other protein, or the modified antibody which is formed by binding to a modifying agent the antibody fragment according to claim 30 or the fused antibody comprising the antibody fragment according to claim 30 fused to a peptide or other protein.

39. A protective or therapeutic agent for cancers and/or viral infectious diseases comprising the antibody fragment according to claim 30, the fused antibody comprising the antibody fragment according to claim 30 fused to a peptide or other protein, or the modified antibody which is formed by binding to a modifying agent the antibody fragment according to claim 30 or the fused antibody comprising the antibody fragment according to claim 30 fused to a peptide or other protein.

40. A method for the manufacture of a medicament for preventing or treating cancers and/or viral infectious diseases comprising using the antibody fragment according to claim 30, the fused antibody comprising the antibody fragment according to claim 30 fused to a peptide or other protein or the modified antibody which is formed by binding to a modifying agent the antibody fragment according to claim 30 or the fused antibody comprising the antibody fragment according to claim 30 fused to a peptide or other protein.


SEQUENCE LISTING (20 sequences)

SEQ ID NO: 1 - protein, 122 aa, Homo sapiens
EVQLLQSGGGLVQPGRSLRLSCAASGFTFDDYAMHWVRQAPGKGLEWVSGISWNGGNIDYADSVRGRFTISRDNAKNSLYLQMDSLRAEDTALYYCAKAPGMLIYYSYMDVWGKGTMVTVSS

SEQ ID NO: 2 - protein, 5 aa, Homo sapiens
DYAMH

SEQ ID NO: 3 - protein, 17 aa, Homo sapiens
GISWNGGNIDYADSVRG

SEQ ID NO: 4 - protein, 13 aa, Homo sapiens
APGMLIYYSYMDV

SEQ ID NO: 5 - DNA, 366 bp, Homo sapiens
gaggtgcagctgttgcagtctgggggaggcttggtacagcctggcaggtctctgagactctcctgtgcagcctctggattcacctttgatgattatgccatgcactgggtccggcaagctccagggaagggcctggagtgggtctcaggtattagttggaatggtggtaacatagactatgcggactctgtgaggggccgattcaccatctccagagacaacgccaagaactccctctatctgcaaatggacagtctgagagctgaggacacggccttgtattactgtgcaaaagcccccggaatgcttatctactactcctacatggacgtctggggcaaagggacaatggtcaccgtctcttca

SEQ ID NO: 6 - protein, 109 aa, Homo sapiens
QPVLTQPPSVSVSPGQTARITCSGDRLPRQYVYWYQQKPGQAPVLLIYKDIERPSGIPERFSGSTSGTTVTLTINGVQAEDEADYSCQSADSSETYPVFGGGTKVTVLG

SEQ ID NO: 7 - protein, 11 aa, Homo sapiens
SGDRLPRQYVY

SEQ ID NO: 8 - protein, 7 aa, Homo sapiens
KDIERPS

SEQ ID NO: 9 - protein, 11 aa, Homo sapiens
QSADSSETYPV

SEQ ID NO: 10 - DNA, 327 bp, Homo sapiens
cagcctgtgctgactcagcccccctcggtgtcagtgtccccaggacagacggccaggatcacctgctctggagatcgattgccaagacaatatgtttattggtaccaacagaagccaggccaggcccctgttttactaatatataaggatattgagaggccctcaggaattcctgagcgattctccggctccacttcagggacaacagtcacgttgaccatcaatggagtccaggcggaagacgaggctgactattcctgtcaatcagcggacagtagtgaaacttatccggtgttcggcggggggaccaaggtcaccgtcctaggt

SEQ ID NO: 11 - protein, 5 aa, Artificial Sequence
  Description of Artificial Sequence Synthetic peptide
GGGGS

SEQ ID NO: 12 - DNA, 15 bp, Artificial Sequence
  Description of Artificial Sequence Synthetic oligonucleotide
ggtggaggcggttca

SEQ ID NO: 13 - protein, 10 aa, Artificial Sequence
  Description of Artificial Sequence Synthetic peptide
GGGGSGGGGS

SEQ ID NO: 14 - DNA, 30 bp, Artificial Sequence
  Description of Artificial Sequence Synthetic oligonucleotide
ggtggaggcggttcaggcggaggtggctct

SEQ ID NO: 15 - protein, 15 aa, Artificial Sequence
  Description of Artificial Sequence Synthetic peptide
GGGGSGGGGSGGGGS

SEQ ID NO: 16 - DNA, 45 bp, Artificial Sequence
  Description of Artificial Sequence Synthetic oligonucleotide
ggtggaggcggttcaggcggaggtggctctggcggtggcggatcg

SEQ ID NO: 17 - DNA, 434 bp, Artificial Sequence
  Description of Artificial Sequence Synthetic polynucleotide
atgcggcccagccggccatggccgaggtgcagctgttgcagtctgggggaggcttggtacagcctggcaggtctctgagactctcctgtgcagcctctggattcacctttgatgattatgccatgcactgggtccggcaagctccagggaagggcctggagtgggtctcaggtattagttggaatggtggtaacatagactatgcggactctgtgaggggccgattcaccatctccagagacaacgccaagaactccctctatctgcaaatggacagtctgagagctgaggacacggccttgtattactgtgcaaaagcccccggaatgcttatctactactcctacatggacgtctggggcaaagggacaatggtcaccgtctcttcaggtggaggcggttcaggcggaggtggctctggcggtggcggatcg

SEQ ID NO: 18 - protein, 144 aa, Artificial Sequence
  Description of Artificial Sequence Synthetic polypeptide
AAQPAMAEVQLLQSGGGLVQPGRSLRLSCAASGFTFDDYAMHWVRQAPGKGLEWVSGISWNGGNIDYADSVRGRFTISRDNAKNSLYLQMDSLRAEDTALYYCAKAPGMLIYYSYMDVWGKGTMVTVSSGGGGSGGGGSGGGGS

SEQ ID NO: 19 - DNA, 391 bp, Artificial Sequence
  Description of Artificial Sequence Synthetic polynucleotide
cagcctgtgctgactcagcccccctcggtgtcagtgtccccaggacagacggccaggatcacctgctctggagatcgattgccaagacaatatgtttattggtaccaacagaagccaggccaggcccctgttttactaatatataaggatattgagaggccctcaggaattcctgagcgattctccggctccacttcagggacaacagtcacgttgaccatcaatggagtccaggcggaagacgaggctgactattcctgtcaatcagcggacagtagtgaaacttatccggtgttcggcggggggaccaaggtcaccgtcctaggtgcggccgcaggtgcgccggtgccgtatccggatccgctggaaccgcgtgccgcatagactgttg

SEQ ID NO: 20 - protein, 127 aa, Artificial Sequence
  Description of Artificial Sequence Synthetic polypeptide
QPVLTQPPSVSVSPGQTARITCSGDRLPRQYVYWYQQKPGQAPVLLIYKDIERPSGIPERFSGSTSGTTVTLTINGVQAEDEADYSCQSADSSETYPVFGGGTKVTVLGAAAGAPVPYPDPLEPRAA